(19) United States
(12) Patent Application Publication
(10) Pub. No.: US 2019/0055512 A1
(43) Pub. Date: Feb. 21, 2019

(54) METHODS AND COMPOSITIONS FOR SUBSTITUTING MEMBRANE LIPIDS IN LIVING CELLS

(71) Applicant: The Research Foundation for The State University of New York, Albany, NY (US)

(72) Inventors: Erwin LONDON, Stony Brook, NY (US); Guangtao LI, Stony Brook, NY (US)

(73) Assignee: The Research Foundation for The State University of New York, Albany, NY (US)

(21) Appl. No.: 16/078,685

(22) PCT Filed: Feb. 21, 2017
(86) PCT No.: PCT/US17/18666

§ 371 Date: Aug. 22, 2018

Related U.S. Application Data
(60) Provisional application No. 62/298,151, filed on Feb. 22, 2016.
(60) Provisional application No. 62/349,964, filed on Jun. 14, 2016.
(60) Provisional application No. 62/424,063, filed on Nov. 18, 2016.

(51) Int. Cl. C12N 5/00; G01N 33/92; G01N 33/58; G01N 33/60

(52) U.S. Cl. CPC C12N 5/0006; G01N 33/60; G01N 33/582; G01N 33/92

(57) ABSTRACT

The current disclosure provides methods, compositions and kits for substitution of cell membrane lipids in living cells. The current methods and compositions further provide methods for the efficient exchange of lipids with the endogenous lipids present in the outer leaflet of the cellular membrane. The methods and compositions of the current disclosure facilitate the exchange of lipids within the cellular membrane with those present in cyclodextrin-lipid complexes, which enables the utilization and analysis of membrane lipid composition, as well as the effect of altering the membrane lipid composition in living cells.

### CROSS REFERENCE TO RELATED APPLICATIONS

This application claims priority from U.S. Provisional Application No. 62/298,151 filed on Feb. 22, 2016, U.S. Provisional Application No. 62/349,964 filed on Jun. 14, 2016, and U.S. Provisional Application No. 62/424,063 filed on Nov. 18, 2016, the entire contents of each of which are incorporated herein by reference.

### STATEMENT REGARDING FEDERALLY SPONSORED RESEARCH

This invention was made with government support under grant numbers GM099892 and GM112638 awarded by the National Institute of Health, and grant number DMR1404985 awarded by the National Science Foundation. The government has certain rights in the invention.

### FIELD OF THE DISCLOSURE

The present disclosure relates to methods for the substitution and exchange of certain membrane lipids. The current disclosure also relates to the preparation of cyclodextrin-lipid complexes. The cyclodextrin-lipid complexes and methods of the present disclosure can be used, for example, in kits for the efficient substitution of endogenous membrane lipids with the lipids bound to cyclodextrin-lipid complexes and the tracking or analysis thereof.

### BACKGROUND

Understanding of the function of the lipids in membranes surrounding cells and their internal compartments has been hindered by the inability to manipulate membrane lipid composition. The eukaryotic plasma membrane exhibits asymmetry (i.e., a difference in inner membrane leaflet and outer membrane leaflet lipid composition) with respect to its lipid distribution across the lipid bilayer. Generally, the outer membrane leaflet in mammalian cells is composed primarily of sphingomyelin (SM) and phosphatidylcholine (PC). In contrast, the inner or cytoplasmic leaflet consists mostly of aminophospholipids, e.g., phosphatidylethanolamine (PE), and phosphatidylserine (PS). Bretscher, M S. Nat. New Biol (1972) 236, pp. 11-12. The asymmetric arrangement of lipids in the cellular membrane affects various biological properties, such as membrane permeability, membrane potential, surface charge, the mechanical stability of membranes, and membrane shape. Hill W. G., et al. J. Gen. Physiol. (1999) 114, pp. 405-414; Hill W. G., Zeidel M. L. J. Biol. Chem. (2000) 275 pp. 30176-30185; Manno S., et al. Proc. Nat. Acad. Sci. USA. (2002) 99 pp. 1943-1948. Therefore, the ability to manipulate the lipid composition of living cell membrane would provide a useful tool for use in research of cell membrane-mediated pathological disease. However, certain classes of lipids, such as phosphatidylinositides (PI) and glycosphingolipids, contain wide variations in headgroup structure, and all classes of lipids can have varying acyl chains, which creates hundreds of membrane lipid species. The sheer volume of membrane lipid species alone creates difficulties in developing methods for altering membrane lipid composition.

Present methods for the manipulation of membrane lipids involve the use of lipid synthesis inhibitors that modulate specific pathways. Delgado A., et al. Biochim Biophys Acta (2006) 1758 pp. 1957-1977. Notably, existing methods include several drawbacks. For example, methods that deploy synthesis inhibitor molecules are slow acting, effective on a limited number of lipids, or not sufficiently lipid-specific. Current metabolic engineering methods are laborious and only effective on bacteria. In addition, the current methods do not permit efficient substitution of a single type of lipid, or the introduction of unnatural or exogenous lipids into the cell.

Lipid substitution represents a promising approach to overcome the foregoing shortcomings. The most widely used lipid exchange agents are cyclodextrins (CDs). However, their use in mammalian cells is generally limited to β-cyclodextrins (βCDs, such as MβCD or HPβCD) for the transfer and modification of cholesterol. For example, when βCDs are added to cells cholesterol is removed from the cellular membrane. In addition, by loading βCD with exogenous cholesterol and then adding sterol-βCD complexes to cells, cholesterol can be delivered into cells. See Kim, J. and London, E. Lipids (2015) 50, pp. 721-734; and Zidovetzki, R., and Levitan, I. Biochimica et biophysica acta (2007) 1768, pp. 1311-1324. However, the use of βCDs to exchange phospholipid or sphingolipids has been limited to the use of methyl β-cyclodextrin (MβCD), and even then has been limited by a very low amount of lipid exchange and the need to replenish cellular cholesterol.

To date, studies of phospholipid or sphingolipid modification has been limited to the use of model membrane vesicles, which mimic the asymmetric lipid distribution seen in the plasma membrane of cells. Common approaches using model membrane vesicles use βCD to exchange lipids between vesicles through the use of lipid-loaded MβCD. See Huang and London, Langmuir (2013) 29, pp. 14631-14638. Briefly, two vesicles having different lipid compositions are incubated with MβCD. The MβCD binds lipids from the vesicles and shuttles the bound lipids between the outer leaflet of the vesicles. MβCD cannot cross membranes, and thus cannot alter lipids in the inner leaflet of the vesicles causing the vesicles to mimic highly asymmetric membranes.

The use of lipid-loaded MβCD to introduce lipids into cells has been attempted, but these studies did not demonstrate exchange of lipids within the cell membrane. See Kainu, V., et al. The Journal of biological chemistry (2008) 283, pp. 3676-3687; and Kainu, V., et al. Journal of lipid research (2010) 51, pp. 3533-3541. Additionally, the use of MβCD resulted in complications during extraction of membrane cholesterol during introduction of the exogenous lipids.

Unlike current methods, the methods and compositions of the present disclosure utilize a separate and distinct class of cyclodextrins, α-cyclodextrins (αCD), which exhibit unique characteristics (e.g., small hydrophobic cavity) that catalyze the exchange of lipids in a cellular membrane without the extraction of membrane cholesterol. Additionally, the present disclosure identifies a subset of α-cyclodextrins, methylα-cyclodextrins (MαCD) that is able to bind lipids at low concentrations and efficiently promote the exchange of cellular phospholipids and sphingolipids from cell plasma membrane outer leaflets of living mammalian cells with exogenous lipids.

### SUMMARY OF THE DISCLOSURE

The present disclosure relates to methods for the substitution of certain membrane lipids in living cells. In certain embodiments of the present disclosure, methods for the exchange of certain membrane lipids in living cells include providing a sample of living cells having a cellular membrane composed of at least one lipid bilayer and incubating at least one cyclodextrin-lipid complex of the present disclosure with the sample of living cells such that incubation results in the exchange of lipids between the outer leaflet of a cellular lipid bilayer and exogenous lipids encompassed in the cyclodextrin-lipid complex. In specific embodiments of the present disclosure, the cyclodextrin included in a cyclodextrin-lipid complex is an alpha-cyclodextrin, specifically a methyl-alpha-cyclodextrin. In preferred embodiments, the methods of the present disclosure result in the formation of living cells that include at least one exogenous lipid in the outer leaflet of the cellular membrane. In another embodiment, the methods of the present disclosure result in the formation of living cells that include at least 70% exogenous lipid in the outer leaflet of the cellular membrane. In a specific embodiment, the methods of the present disclosure result in the formation of living cells that include an outer leaflet composed entirely of exogenous lipids.

The current disclosure also relates to the preparation of cyclodextrin-lipid complexes for use in exchanging membrane lipids in living cells. In some embodiments, the cyclodextrin included in a cyclodextrin-lipid complex is an alpha-cyclodextrin or a methyl-alpha-cyclodextrin. In certain embodiments, the cyclodextrin molecule of a cyclodextrin-lipid complex binds at least one exogenous lipid. In other embodiments, the cyclodextrin molecule binds an exogenous lipid at a hydrophobic interior portion of the cyclodextrin molecule. In specific embodiments, the lipid bound to a cyclodextrin-lipid complex is a sphingolipid or a phospholipid. In specific embodiments, the lipid is sphingomyelin or phosphatidylcholine and/or derivatives thereof. In yet another embodiment, the lipid is an unnatural lipid, such as N-hepadecanoyl-D-erythro-sphingosylphosphorylcholine (C_{17:0} SM). In some embodiments, the lipid bound in a cyclodextrin-lipid complex is labeled such that it can be identified and tracked, such as for example, radio-labeled or fluorescent dye labeled lipids.

In another aspect of the present disclosure, kits for the exchange of membrane lipids in living cells are provided. The cyclodextrin-lipid complexes and methods of the present disclosure can be used, for example, in kits for efficient replacement of cell membrane lipids with exogenous lipids. In preferred embodiments, kits of the present disclosure include an amount of an alpha-cyclodextrin, specifically a methyl-alpha-cyclodextrin. In certain embodiments, kits of the present disclosure include at least one exogenous lipid. In specific embodiments, the exogenous lipid is a sphingolipid or a phospholipid. In other embodiments, the exogenous lipid is sphingomyelin or phosphatidylcholine and/or derivatives thereof. In yet another embodiment, the lipid is an unnatural lipid, such as N-hepadecanoyl-D-erythro-sphingosylphosphorylcholine (C_{17:0} SM). In some embodiments, the lipid provided is labeled such that it can be identified and tracked, such as for example, radio-labeled or fluorescent dye labeled lipids. In other embodiments, kits of the present disclosure include a cell sample. In certain embodiments, the cell sample includes labeled cell membrane lipids. In some instances, the kits of the present disclosure include fluorescent dyes, or radio-isotopes and instructions for incorporating the same in a cyclodextrin-lipid complex. In preferred embodiments, the kit includes instructions for monitoring the exchange of lipids between cyclodextrin-lipid complexes and cells and/or quantifying such an exchange.

### BRIEF DESCRIPTION OF THE DRAWINGS AND TABLES

The file of this patent contains at least one drawing executed in color. Copies of this patent with color drawing(s) will be provided by the Patent and Trademark Office upon request and payment of the necessary fee.

FIG. 1. A schematic illustration of an exemplary lipid exchange method of the present disclosure. The exogenous lipids (red) are incubated to form multilamillar vesicles and then incubated with an alpha-cyclodextrin (e.g., MαCD) to form a cyclodextrin-lipid complex (hexagons) that facilitates the exchange of exogenous lipids (red) between a cyclodextrin-lipid complex and the lipids in the outer leaflet of the plasma membrane of a cell (blue). If the exogenous lipid on the cyclodextrin-lipid complex is in excess, the outer leaflet composition of the cell membrane will be entirely replaced by exogenous lipids bound to the cyclodextrin-lipid complex. When endogenous cellular lipid (blue) is radio labeled, the exchange of lipids between cyclodextrin-lipid complexes and the cell membrane can be observed by a loss of cell associated endogenous (labeled) lipid.

FIGS. 2A-C. The kinetics of lipid exchange. The effects of incubation with cyclodextrin-lipid complexes and A549 cells at 37° ° C., as well as temperature and time dependence of the exchange methods. (A) Lipid exchange kinetics. Removal of endogenous sphingomyelin (SM) was measured by ³H-labeled SM remaining in the cells and compared to the amount of fluorescently labeled 1,2-dipalmitoyl-sn-glycero-3-phosphoethanolanamine-N-(7-nitro-2-1,3-benzoxadiazol-4-yl) (NBD-DPPE) lipid delivered to the cells. Half-time is 10-15 minutes. (B) Effect of exchange temperature. At elevated temperature (37° C.), less labeled SM were detected in cell membranes, indicating that increasing incubation temperature from 15° C. to 37° C. is important for efficient exchange of lipids using the present methods. (C) Dependence of % ³H incubation time on SM exchange. At all incubation times tested, 30 minute incubation, 30+30 minute incubation or 60 minute incubation, lipid exchange was not significantly different. For the 30+30 minute condition, after an initial 30 minute incubation of cells with labeled lipid, supernatant was removed and replaced with fresh cyclodextrin-lipid complex containing 1.5 mM SM loaded with 40 mM MαCD, and cells were incubated for a second 30 minute interval. Data was normalized to the level of radioactive PS and PI in the same sample. Averages and standard deviations of 3 experiments are shown.

FIGS. 3A-B. Localization of exogenous lipids after exchange. (A) Shows a superposition of a bright field image of the cell and a fluorescent confocal slice image of cells after 1 hour lipid exchange with 1:9 NBD-DPPE:brain SM (bSM) lipids bound to 40 mM MαCD (left) without dithionite treatment, or followed by incubation with dithionite for 5 minutes (center) or 10 minutes (right). Together, showing that exchanged lipid remains in the cellular membrane. (B) Temperature dependence of fraction of exchanged lipid residing in the cellular membrane. 1.5 mM 1:9 NBD-DPPE:bSM bound to 40 nmM MαCD in a cyclodextrin-lipid complex was delivered to cells in a 1 hour lipid exchange at 37° C. Then the amount of NBD-DPPE in the outer leaflet of the plasma membrane was assayed using dithionite. Dithionite destroys NBD-fDPPE groups exposed on the outer leaflet of the plasma membrane, and thus eliminates NBD-DPPE fluorescence. Dithionite would not affect NBD-lipid fluorescence that has reached the plasma membrane inner leaflet or interior of the cell.

FIGS. 4A-B. Effect of the concentration of methyl αCD and lipid mixed with methyl αCD on lipid exchange. The amount of radioactive endogenous SM replaced in A549 cells by lipid exchange with cyclodextrin-lipid complexes was detected. (A) The effect of MαCD concentration on residual endogenous (³H-labeled) SM percentage was detected in cellular membranes post lipid exchange using the present methods. The lipid concentration here was 1.5 mM bSM for each concentration of methyl αCD tested. (B) The effect of lipid concentration on residual endogenous (3H-labeled) SM percentage detected in cellular membranes post lipid exchange. The lipid concentration here varied while MαCD concentration remained constant at 30 mM for all SM concentrations tested. Data was normalized to the level of radioactive PS and PI in the same sample. Averages and standard deviations of 3 experiments are shown. Data shows that lipid exchange with at least 30 mM MαCD is effective at almost all lipid concentrations tested, with about 75-80 percent of endogenous lipids being replaced.

FIGS. 5A-C. Efficient exchange is specific to outer membrane leaflet lipids. (A-B) Show histograms identifying the removal of radiolabeled endogenous lipids from A549 cells upon lipid exchange with non-radioactive lipid (1.5 mM bSM bound to 40 mM MαCD) from cyclodextrin-lipid complexes. Only SM shows a high % exchange because SM is predominantly located in plasma membrane outer leaflet, not internal compartments of cells. Other lipids, e.g., PS, PI, PC, PE exhibited little to no exchange either because they are in the inner leaflet, or internal membranes. As such, the exchange methods of the present disclosure are highly specific. (A) 100% indicates the endogenous radio-labeled lipid value before exchange. Measurements normalized to 100% value of each lipid assuming that PS+PI levels are the same before and after exchange. (B) Percent of phospholipid orsphingolipid radioactivity relative to that before exchange was calculated for samples with MαCD (after exchange). Values for A-B are normalized to 100% before exchange for each lipid. (C) Charred thin-layer chromatography (TLC) detection of cellular lipids after 1 hour exchange with MαCD-(SM or POPC) lipid complexes. Lane 1 shows control samples with no MαCD and noexogenous lipids. Lane 2 shows control condition having 1.5 mM exogenous bSM with no MαCD. Lane 3 shows lipid exchange with 1.5 mM exogenous bSM and MαCD, while lane 4 shows lipid exchange with 3 mM POPC and MαCD. Again showing that outer leaflet lipids were exchanged.

FIGS. 6A-C. Outer membrane leaflet exchange specificity and efficiency. (A) The composition of cellular lipids extracted from A549 cells after exchange with 1.5 mM bSM complexed with 40 mM MαCD was determined by measuring the radioactivity of samples of extracted lipids post exchange. Background (treatment without MαCD) levels were removed from all conditions. (B)^{14C} radio-labeled SM was exchanged into cells by 1 hour exchange incubation with 1.5 mM bSM complexed with 40 mM MαCD. Then ¹⁴C radio-labeled SM was exchanged back out of the cells either immediately after the initial exchange (0) or 1 hour after the initial exchange into cells (1). Second exchange was feasible and no difference was seen in lipid exchange immediately after exchange or 1 hour later. (C) Cells were harvested immediately after exchange with 1.5 mM bSM complexed with 40 mM MαCD (0), 2 hours after exchange (2), 4 hours after exchange (4) or 6 hours post exchange (6), and analyzed by TLC in order to determine if lipid exchange was maintained and/or increased over time. Membrane asymmetry remained stable over time without significant change.

FIGS. 7A-B. Lipid exchange by αCD was efficient across many cell types and for many different lipids. (A) The data herein show the extent of lipid exchange between exemplary cell lines tested in the present disclosure. Radioactive SM was replaced after lipid exchange with MαCD-SM cyclodextrin-lipid complexes of the present disclosure and detected in all cell types tested. Between 75% and 60% SM outer leaflet lipid was exchanged in all cell types, showing that the present methods are highly efficient in all cell types. (B) Radioactive cellular SM was replaced by lipid exchange with MαCD-lipid complexes of the present disclosure. Non-radioactive lipids bound to MαCD are as follows: bSM=brain sphingomyelin; eSM=egg sphingomyelin; POPC=1-palmitoyl 2-oleoyl phosphatidylcholine, DOPC=dioleoyl phosphatidylcholine. Lipid concentrations as listed. MαCD concentration 40 mM. Between 70% and 80% of cellular endogenous SM was removed using exogenous bSM, exogenous POPC, exogenous bSM/POPC, or exogenous bSM/DOPC with the present methods.

FIGS. 8A-B. Lipid exchange does not alter cellular function. (A) Images of untreated cells, cells that underwent cholesterol depletion for 30 minutes with 10 mM MβCD at 37° C., and cells that were subject to SM and POPC lipid exchange using the present methods were taken after treatment with TF-AF488 (TF) for 10 minutes at room temperature to determine endocytosis levels for all conditions. Green is TF-AF488 staining and Blue is cell membrane staining with CellMask™. Scale bar 50 nm. (B) Shows quantification of TF endocytosis in all conditions tested. Average values and standard deviations from 3 separate experiments are shown. TF endocytosis was comparable to that of cells that did not undergo lipid exchange, and thus cellular function is not altered by the present exchange methods.

FIGS. 9A-B. Endogenous membrane lipids were successfully radio-labeled with ³H. (A) Distribution of endogenous radio-labeled membrane lipids was determined by TLC. Here, the sample was fractionated and radioactivity was measured in the lipid bands by scintillation counting. Background radioactivity is shown (numbered fractions). (B) TLC showing abundance of each membrane lipid charred for quantitative analysis. Two dimensional TLC shows that all membrane lipids PS, PI, SM, PC and PE were successfully separated and their relative abundance was calculated. Together, the data show that PC is most abundant; PE, PS+PI, and SM are present to a lesser extent, which is consistent with their relative abundance in cell membranes as judged by radioactivity as shown in FIG. 9A.

FIG. 10A-B. Cell membrane outer leaflet lipid exchange was consistent with ganglioside exchange. (A) Cells were subjected to bSM or bSM/POPC lipid exchange with 40 mM MαCD for either 30 mins or 60 mins at 37° C., and then 30 minute incubation with FITC-labeled cholera toxin B (CTxB). Green is FITC staining and Blue is cell membrane staining with CellMask™. Scale bar 50 nm. (B) Quantitative analysis of FITC labeled cholera toxin B binding to cells. Averages and standard deviations from 3 separate experiments are shown.

### TABLE 1. EFFECT OF LIPID EXCHANGE ON CELL PHOSPHOLIPID AND SPHINGOMYELIN CONTENT

Mass spectrometry (MS) data was obtained and is shown as the average of duplicate experiments. Radio labeling and TLC experiments represent the average and standard deviation from 3 different experiments. ^{a}total lipids (phospholipids+sphingomyelin) not corrected for trace amounts of PG and CL seen in MS runs. ^{b}values shown after lipid exchange are percent of remaining endogenous lipid. ^{c}radioactivity is the sum of PS and PI lipids. ^{d}exogenous radio labeled SM was 37.9±0.4% of the total lipid after exchange. SM exchange efficiency was between 70% and 81%.

### TABLE 2. TIME DEPENDENT EFFECT OF LIPID EXCHANGE ON CELLULAR MORPHOLOGY

Cells were sensitive to treatment with MαCD alone after about 15 minutes of incubation. However, when MαCD was pre-incubated with lipid vesicles prior to incubation with cells cells maintained normal morphology after incubation and lipid exchange.

### TABLE 3. PERCENT OF ABUNDANT (GREATER THAN 1%) PHOSPHOLIPIDS IN CELLS BEFORE AND AFTER LIPID EXCHANGE

The average from duplicate experiments is shown. The p values show the significance of the differences between species group averages for each species headgroup type. ^{a} ratio (% lipid species in cells before exchange/after exchange). A higher ratio is observed for a specific acyl chain species when it is preferentially removed during exchange relative to average species group, e, ether lipid; p, ether lipid with double bond between C1 and C2.

### TABLE 4. PERCENT OF SPHINGOMYELIN SPECIES PRESENT IN UNTREATED CELLS AND CELLS THAT UNDERGO METHYL αCD MEDIATED LIPID EXCHANGE

Ratio 1, % of total SM before exchange/after exchange. The higher ratio values indicate an increased percent of endogenous SM after exchange. Total lipids, phospholipids plus sphingomyelin.

### DETAILED DESCRIPTION OF THE DISCLOSURE

The present disclosure is directed to the development and use of lipid-bound cyclodextrins to efficiently exchange lipids present in the outer leaflet of a cell membrane bilayer. In certain aspects of the present disclosure, exogenous lipids are bound to cyclodextrin molecules to form cyclodextrin-lipid complexes capable of exchanging the exogenous lipids bound thereto with endogenous lipids located in the outer leaflet of a cell membrane without harming the cell. Without being bound by a particular theory, the present methods are premised on the discovery that certain cyclodextrins are capable of exchanging lipids between their uniquely sized and shaped hydrophobic core and the outer leaflet of the cellular membrane without depleting cellular cholesterol, and thus cells remain viable after lipid exchange.

### Definitions

The term “cyclodextrin” or “CD” as used herein refers to a family of cyclic oligosaccharides, composed of five or more α-D-glucopyranoside units. More specifically, cyclodextrins (CDs) are cyclic oligomers of glucose having, for example, six (α-cyclodextrins, αCDs), seven (β-cyclodextrins, βCDs), or eight (γ-cyclodextrins, γCDs) glucose units. Cylclodextrins include a hydrophobic interior portion (cavity) capable of binding hydrophobic molecules. Cyclodextrins of the present disclosure include a lipophilic central cavity and a hydrophilic outer surface. Examples of cyclodextrins which can be incorporated in the cyclodextrin-lipid complexes of the present disclosure include, but are not limited, α-cyclodextrins, β-cyclodextrins and γ-cyclodextrins, as well as substituted cyclodextrins. Non-limiting examples of β-cyclodextrins include methyl-beta-cyclodextrin, carboxymethyl-beta-cyclodextrin, hydroxypropyl-beta-cyclodextrin and tetradecasulfated-beta-cyclodextrin, γ-cyclodextrins of the present disclosure can include, for example, carboxyethyl-γ-cyclodextrin, hydroxypropyl-γ-cyclodextrin, acetyl-γ-cyclodextrin, carboxymethyl-γ-cyclodextrin, succinyl-γ-cyclodextrin, 2-hydroxyethyl-γ-cyclodextrin, ethyl-γ-cyclodextrin, n-butyl-γ-cyclodextrin. In a preferred embodiment of the present disclosure, the cyclodextrin is an α-cyclodextrin. Non-limiting examples of α-cyclodextrins include methyl-α-cyclodextrins (e.g., a species of α-cyclodextrins with a methyl group or methyl groups attached to the glucose rings of a cyclodextrin, such as dimethyl-α-cyclodextrin and randomly methylated alpha cyclodextrins), sulfo-α-cyclodextrin, and hydroxypropyl-α-cyclodextrin, carboxyethyl-α-cyclodextrin, succinyl-α-cyclodextrin, hydroxyethyl-α-cyclodextrin, ethyl-α-cyclodextrin, and n-butyl-α-cyclodextrin.

The term “cyclodextrin-lipid complex” or “CD-lipid complex” as used herein refers to a complex that is formed between a lipid and at least one cyclodextrin whereby the lipid or lipids are bound to the cyclodextrin(s) (at their hydrophobic interior cavity). In certain embodiments, a cyclodextrin-lipid complex includes a plurality of cyclodextrin molecules bound to a lipid. In a preferred embodiment, a cyclodextrin-lipid complex of the present disclosure includes a lipid bound to a single cyclodextrin molecule.

The term “binding”, “to bind”, “binds, “bound” or any derivation thereof refers to any direct interaction, e.g., chemical bond, between two or more molecules, including, but not limited to, covalent bonding, ionic bonding, and hydrogen bonding. Thus, this term encompasses the interaction between a cyclodextrin and a lipid. More specifically, the interaction between the hydrophobic core of a cyclodextrin and a lipid, e.g., sphingolipid and/or phospholipid.

The term “lipid” or “lipids” used herein refers to an organic molecule that is insoluble in water and soluble in non-polar solvents. Lipids include fatty acids, esters derived from a fatty acid and a long-chain alcohol, triacylglycerol, phospholipids, prostaglandin, sphingolipids, and sterols. Lipids of the present disclosure can be, for example, labeled, such as lipids labeled with a fluorescent dye, or incorporate a radioactive isotope (e.g., ¹⁴C or ³H). Lipids can be a naturally occurring lipid that has been created synthetically or isolated from cells. In some embodiments, the lipids of the present disclosure can be an “unnatural lipid”, or a lipid that is not found in nature. Unnatural lipids include, for example, lipids with a modified acyl chain, length(s), composition, function or a combination thereof when compared to its naturally occurring (unmodified) counterpart, such as, for example, N-hepadecanoyl-D-erythro-sphingosylphosphorylcholine (C_{17:0} SM). In some instances, unnatural lipids include lipid analogs that are modified in such a manner that they are not subject to phospholipase mediated enzymatic activity.

“Sphingolipids” as used throughout the present disclosure means a class of lipids derived from the aliphatic amino alcohol sphingosine. The sphingosine backbone is O-linked to a charged head group such as, for example, ethanolamine, serine, or choline. The sphingosine backbone is also amide-linked to an acyl group, such as a, fatty acid. Sphingolipids can be found, for example, in neural cells. Non-limiting examples of sphingolipids include ceramides, sphingomyelins, and glycosphingolipids. Ceramides consist of a fatty acid chain attached to a sphingosine backbone by an amide linkage. Sphingomyelins (SM) contain a phosphocholine or to the 1-hydroxy group of a ceramide. Other sphingolipids can have phosphoethanolamine or phosphoinositol esterified to a ceramide. Glycosphingolipids are ceramides with one or more sugar residues joined by β-glycosidic linkage at the 1-hydroxyl position. Glycosphingolipids include cerebrosides and gangliosides. Simple cerebrosides have a single glucose or galactose at the 1-hydroxy position. Others have two sugars attached, and globosides can have more than two. Gangliosides have at least three sugars, one of which must be sialic acid. Sphingolipids are generally present in the outer leaflet of the plasma membrane lipid bilayer. In preferred embodiments of the present disclosure, sphingolipids are sphingomyelins or derivatives thereof.

“Phospholipids” as used herein means a class of lipids that contain a phosphate group attached to two fatty acid chains by a glycerol molecule. The phosphate group forms a negatively-charged polar head, which is hydrophilic. In certain embodiments, the net charge of a lipid can be neutral when the polar group attached to the phosphate group by a phosphoester is positively charged a positive charge. The fatty acid chains form uncharged, non-polar tails, which are hydrophobic. Non-limiting examples of phospholipids of the present disclosure are those present in the outer leaflet of the cell membrane, such as phosphatidylcholine (PC). Phosphatidylcholines likely to be in the outer leaflet include 1-dioleoyl phosphatidylcholine (DOPC), 1-palmitoyl 2-oleoyl phosphatidylcholine (POPC) and 1-stearoyl-2-oleoyl-phosphatidylcholine (SOPC). Additional PCs that are present in membranes would be analogous to those above, but with linoleic acid, linolenic acid, arachidonic acid or docosahexenoic acid in the 2 position. In certain instances, these latter species can be found in the inner leaflet, but in the absence of methods that can accurately analyze lipid asymmetry. Phospholipids of the present disclosure also include those present in the inner leaflet of the cell membrane, such as aminophospholipids (e.g., phosphatidylethanolamines (PE), and phosphatidylserine (PS), and phosphatidylinositol (PI) and derivatives thereof.

The term “cell membrane”, “cellular membrane” or “plasma membrane” as used herein refers to the component of a cell surrounding the cytosol that encases the cells contents (e.g., organelles). Cell membranes are composed primarily of lipids, such as phospholipids and sphingolipids, proteins (e.g., transmembrane), and cholesterol. In a preferred embodiment, cell membranes of the present disclosure are eukaryotic cell membranes composed of an asymmetric lipid bilayer, which includes an inner leaflet and an outer leaflet of membrane lipids.

### Compositions

Another aspect of the present disclosure includes the formation of cyclodextrin-lipid complexes for use in the efficient exchange of lipids in living cells. Generally, the cyclodextrin-lipid compositions of the present disclosure are formed by mixing phospholipids and/or sphingolipids in a solvent (e.g., an organic solvent). The lipids are then dried to remove the solvent (e.g., nitrogen or vacuum). The dried lipids are mixed with an aqueous buffer, such as PBS or medium, to form multilamellar vesicles (MLV). The mixture of cyclodextrin and MLV are then incubated together to form cyclodextrin-lipid complexes. Without being bound by any one particular theory, during the incubation step, lipids separate from the MLV and bind to the hydrophobic interior cavity of a cyclodextrin molecule to form a cyclodextrin-lipid complex. Notably, certain cyclodextrins, namely α-cyclodextrins, have a unique hydrophobic cavity that is too small to bind cholesterol, which enables α-cyclodextrin to bind cell membrane lipids, but not sterols (i.e., cholesterol). Therefore, the compositions herein are capable of exchanging lipids with the cellular membrane without removing cholesterol from the cell during the exchange process.

In certain embodiments, cyclodextrin-lipid complexes of the present disclosure include α-cyclodextrin. In some specific embodiments, the alpha-cyclodextrin is a dimethyl-α-cyclodextrin, sulfo-α-cyclodextrin, and hydroxypropyl-α-cyclodextrin, carboxyethyl-α-cyclodextrin, succinyl-α-cyclodextrin, hydroxyethyl-α-cyclodextrin, ethyl-α-cyclodextrin, or n-butyl-α-cyclodextrin. In yet another embodiment, the cyclodextrin is hydroxypropyl-α-cyclodextrin. In a preferred embodiment, the cyclodextrin used to form a cyclodextrin-lipid complex of the present disclosure is methyl-α-cyclodextrin.

As stated above, in certain embodiments the lipids bound to cyclodextrin are lipids commonly found in the cell membrane such as, for example, lipids of the outer leaflet of the plasma membrane. For example, any lipid that includes a polar head group and acyl chain(s) can be used to form cyclodextrin-lipid complexes of the present disclosure. In specific embodiments, the lipids are exogenous phospholipids or sphingolipids. In preferred embodiments of the present disclosure, the sphingolipid is a sphingomyelin or a derivative thereof. In specific embodiments of the present disclosure, the phospholipids is phosphatidylcholine (PC) or a derivative thereof.

In some embodiments, the lipids are brain sphingomyelin (bSM), egg sphingomyelin (eSM), milk sphingomyelin (mSM), 1-palmitoyl-2-oleoyl-sn-glycerol-3-phosphocholine (POPC), 1,2-dioleoyl-sn-glycero-3-phosphocholine (DOPC), phosphatidylethanolamine (PE), phosphatidylserine (PS), 1-palmitoyl-2-oleoyl-sn-glycero-3-phosphoethanolamine (POPE), 1-stearoyl-2-oleoyl-phosphatidylcholine (SOPC) and -palmitoyl-2-oleoyl-sn-glycero-3-phospho-L-serine (POPS), 1,2-dipalmitoyl-sn-glycero-3-phosphoethanolaamine-N-(7-nitro-2-1,3-benzoxadiazol-4-yl) (NBD-DPPE), NBD-SM or NBD-POPC.

In a specific embodiment, the lipids incorporated in cyclodextrin-lipid complexes of the present disclosure are SM (e.g., bSM, eSM, mSM), or a phosphatidylcholine (e.g., POPC, SOPC, DOPC) or derivatives thereof.

In certain embodiments, the lipids incorporated in cyclodextrin-lipid complexes of the present disclosure are endogenous cell membrane lipids (SM, PC, PE, PS), which have been extracted from the plasma membrane of cells, and isolated prior to incorporation in the cyclodextrin-lipid complexes of the present disclosure.

In yet another embodiment, lipids incorporated in the cyclodextrin-lipid complexes of the present disclosure are modified (e.g., labeled) in such a manner that enables the exogenous lipid to be identified. For example, labeled lipids may be identified or detected by any means known to one of ordinary skill in the art, e.g., nuclear magnetic resonance, fluorescence spectroscopy, fluorescent microscopy, mass spectrometry, or chromatography, such as, thin-layered chromatography (TLC) and high-performance TLC (HPTLC).

In a specific embodiment, the lipids are radio-labeled lipids. For example, isolated lipids or cells containing endogenous lipids can be incubated with a solution containing sodium acetate and ³H acetate, e.g., 1.8M sodium acetate and 10 μCi ³H acetate in 10 mL RPMI 1640 medium, for about 24 hours to facilitate the labeling of the lipids. Where the lipids are endogenous lipids contained in cells, after incubation the medium is removed and the cells washed and the labeled lipids can be isolated using known methods, such as lipids extraction with 3:2 (v:v) hexane/isopropanol with vortexing, and then dried. The lipids can then be incubated with alpha-cyclodextrin to form the cyclodextrin-lipid complexes of the present disclosure.

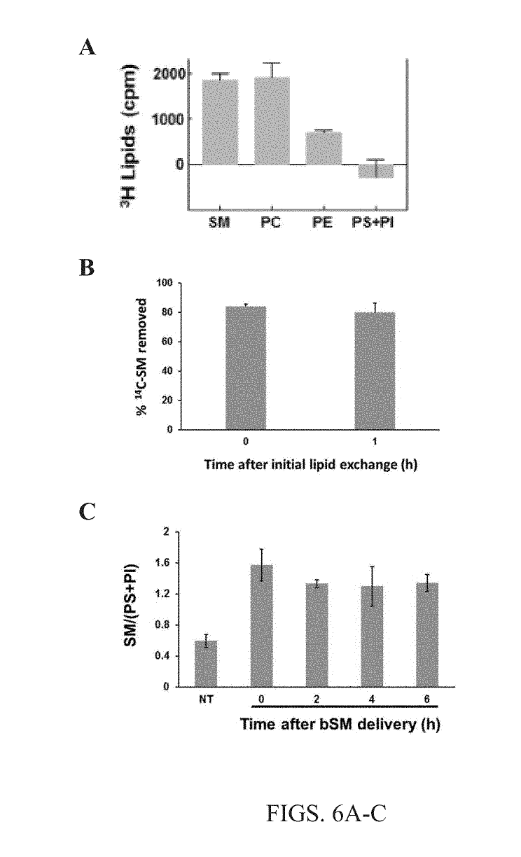

In one instance, the radio-labeled lipid is, for example, ¹⁴C-labeled sphingolipid (e.g., sphingomyelin) or a ¹⁴C-labeled phospholipid (e.g., PC, including POPC, SOPC, DOPC). By way of example, to form a cyclodextrin-lipid complexes of the present disclosure, that includes a radiolabeled lipid, α-cyclodextrin is incubated the lipids to be exchanged (e.g., SM, POPC, or a combination thereof) and 0.5×10⁶ cpm ¹⁴C-SM. After incubation of the α-cyclodextrin and lipid/radio-labeled lipid solution for approximately 30 minutes at 37° C. the radiolabeled ¹⁴C-SM binds to the alpha cyclodextrin forming cyclodextrin-lipid complexes of the present disclosure, which can be used to track the exchange of lipids in living cells using the methods of the instant disclosure as shown in FIG. 6B.

In another example, the lipids are fluorescent dye-labeled lipids. Here, lipids are incubated with a fluorescent dye, such as (7-nitro-2-1,3-benzoxadiazol-4-yl) (NBD), or those described in T. Baumgart, et al., Proc. Natl. Acad. Sci. USA, (2007)104, pp. 3165-3170, the entire contents of which is incorporated herein by reference. These, fluorescent-labeled lipids, such as 1,2-dipalmitoyl-sn-glycero-3-phosphoethanolamine-N-(7-nitro-2-1,3-benzoxadiazol-4-yl) (NBD-DPPE), NBD-SM and NBD-POPC, are then isolated using known methods and then dried. The fluorescent dye-labeled lipids can then be incubated with alpha-cyclodextrin to form the cyclodextrin-lipid complexes of the present disclosure.

In yet other embodiments, the lipids included in the cyclodextrin-lipid complexes of the present disclosure include unnatural lipids, such as, for example, unnatural sphingolipids or phospholipids, with unnatural fatty acids, including those with odd carbon number acyl chains, or deuterium attached to carbon in place of H hydrogen. In specific embodiments, the unnatural phospholipids are unnaturally occurring sphingomyelins. Certain subsets of sphingomyelins, such as brain-SM (bSM) are difficult to track during exchange by, for example, mass spectrometry, with cellular lipids as shown in Table 1, 3 and 4. Therefore, unnaturally occurring SM lipids, e.g. those with unnatural acyl chain lengths, have identified for incorporation in the cyclodextrin-lipid complexes of the present disclosure in order to easily identify and quantify SM lipid exchange. In a specific embodiment, the unnatural lipid bound to an alpha-cyclodextrin in a cyclodextrin-lipid complex of the present disclosure is N-hepadecanoyl-D-erythro-sphingosylphospsphoiylhocholine (C_{17:0} SM).

In some embodiments of the present disclosure, the cyclodextrin-lipid complexes of the present disclosure include an α-cyclodextrin and a phospholipid, sphingolipid or combination thereof. In other embodiments of the present disclosure, the cyclodextrin-lipid complex includes an α-cyclodextrin and at least one sphingolipid. In yet other embodiments, the cyclodextrin-lipid complex includes an α-cyclodextrin bound to sphingomyelin or a derivative thereof. In specific embodiments of the present disclosure, the cyclodextrin-lipid complex includes an α-cyclodextrin and at least one phospholipid. In other embodiments, the cyclodextrin-lipid complex includes an α-cyclodextrin bound to phosphatidylcholine (PC) or a derivative thereof. In certain embodiments, the cyclodextrin-lipid complexes of the present disclosure include a α-cyclodextrin bound to PC, POPE, POPS, POPC, DOPC or a derivative thereof. In specific embodiments of the present disclosure, the cyclodextrin-lipid complex includes an α-cyclodextrin bound to at least one phospholipid and at least one sphingolipid. In other embodiments, the cyclodextrin-lipid complexes of the present disclosure include a α-cyclodextrin bound to SM and POPC, SM and DOPC, or SM and POPE.

In preferred embodiments of the present disclosure, the cyclodextrin-lipid complexes of the present disclosure include a methyl-α-cyclodextrin and a phospholipid, sphingolipid or combination thereof. In specific embodiments of the present disclosure, the cyclodextrin-lipid complex includes a methyl-α-cyclodextrin and at least one phospholipid. In other embodiments, the cyclodextrin-lipid complex includes a methyl-α-cyclodextrin bound to phosphatidylcholine (PC) or a derivative thereof. In certain embodiments, the cyclodextrin-lipid complexes of the present disclosure include a methyl-α-cyclodextrin bound to POPE, POPS, POPC, DOPC or a combination therefore. In certain embodiments, the cyclodextrin-lipid complexes of the present disclosure include a methyl-α-cyclodextrin bound to POPC and DOPC, or POPC and POPE, or POPC and POPS. In other embodiments, the methyl-α-cyclodextrin is bound to POPE and POPS or POPE and DOPC.

In other embodiments of the present disclosure, the cyclodextrin-lipid complex includes a methyl-α-cyclodextrin and at least one sphingolipid. In yet other embodiments, the cyclodextrin-lipid complex includes a methyl-α-cyclodextrin bound to sphingomyelin or a derivative thereof. In specific embodiments, the cyclodextrin-lipid complex includes a methyl-α-cyclodextrin bound to brain-sphingomyelin (bSM) or egg-spingomyelin (eSM) a combination thereof.

In some embodiments, the cyclodextrin-lipid complexes of the present disclosure include a methyl-α-cyclodextrin bound at least 3 lipids. In one embodiment, the cyclodextrin-lipid complexes of the present disclosure include a methyl-α-cyclodextrin bound to any three of the following lipids PC, PS, PI, PE, POPC, POPE, POPS and SM. In specific embodiments, the cyclodextrin-lipid complexes of the present disclosure include a methyl-α-cyclodextrin bound to POPC, POPE and POPS. In other specific embodiments, a methyl-α-cyclodextrin is bound to at least two phospholipids (e.g., PC, PS, PI, PE, POPC, POPE and POPS) and a syphingolipid (e.g., SM).

In specific embodiments of the present disclosure, the cyclodextrin-lipid complex includes a methyl-α-cyclodextrin bound to at least one phospholipid and at least one sphingolipid. In other specific embodiments, a methyl-α-cyclodextrin is bound to at least one phospholipid (e.g., PC, PS, PI, PE, POPC, POPE and POPS) and a sphingomyelin (e.g., bSM, eSM). In specific embodiments, the cyclodextrin-lipid complexes of the present disclosure include a methyl-α-cyclodextrin bound to SM and PC. In other embodiments, the cyclodextrin-lipid complexes of the present disclosure include a methyl-α-cyclodextrin bound to SM and POPC, SM and DOPC, or SM and POPE. In yet another embodiment, the cyclodextrin-lipid complexes of the present disclosure include a methyl-α-cyclodextrin bound to SM and POPC, SM and DOPC, or SM and POPE.

### Methods

Conventional lipid delivery procedures generally involve the use of artificial membrane vesicles and high concentrations β-cyclodextrins. Notably, when vesicles and β-cyclodextrins are incubated with cells, endogenous membrane cholesterol is extracted leading to aberrant levels of cellular cholesterol after incubation—a phenomenon that is toxic to living cells. Additionally, pre-existing lipid delivery methods are inefficient, requiring cyclodextrin treatment for several hours to deliver small amounts of exogenous lipids to a cell, with lowest efficiency for lipids that are most common.

The methods of the current disclosure provide a lipid exchange process by which a lipid is bound to a cyclodextrin to form a cyclodextrin-lipid composition (i.e., cyclodextrin-lipid complex). Cyclodextrin-lipid complexes are then incubated with cells under certain conditions in order to facilitate the efficient exchange of the lipids bound to the cyclodextrin-lipid complexes and the endogenous membrane lipids located within the cellular membrane.

The lipid exchange methods of the present disclosure generally include the formation and use of cyclodextrin-lipid complexes, as described above. More specifically, the present methods include the formation and use of cyclodextrin-lipid complexes composed of an alpha-cyclodextrin and a lipid. In a particularly exemplary method of the present disclosure the lipid-exchange methods of the present disclosure include the formation and use of cyclodextrin-lipid complexes composed of a methyl-alpha-cyclodextrin and a lipid.

In certain embodiments, the cyclodextrins are an alpha-cyclodextrin. As noted above, α-cyclodextrins have a unique structure that provides a unique capability to bind certain lipids, but not sterols (i.e., cholesterol). Specifically, ca-cyclodextrins have a smaller hydrophobic cavity compared to other classes of cyclodextrin, such as β-cyclodextrin and γ-cyclodextrin, which prohibits sterol binding, and thus cell death. In certain embodiments, the alpha-cyclodextrin is a dimethyl-α-cyclodextrin, sulfo-α-cyclodextrin, and hydroxypropyl-α-cyclodextrin, carboxyethyl-α-cyclodextrin, succinyl-α-cyclodextrin, hydroxyethyl-α-cyclodextrin, ethyl-α-cyclodextrin, and n-butyl-α-cyclodextrin.

In a preferred embodiment, the cyclodextrin used to form a cyclodextrin-lipid complex of the present disclosure is methyl-α-cyclodextrin. In yet another embodiment, the cyclodextrin is hydroxypropyl-α-cyclodextrin.

In certain embodiments of the present disclosure, the lipids incorporated in cyclodextrin-lipid complexes are lipids commonly found in the outer leaflet of the cell membrane. For example, any lipid that includes a polar head group and acyl chain(s) can be used to form cyclodextrin-lipid complexes of the present disclosure. In specific embodiments, the lipids used for exchange are phospholipids or sphingolipids. In preferred embodiments of the present disclosure, the sphingolipid is a sphingomyelin or a derivative thereof. In specific embodiments of the present disclosure, the phospholipid is phosphatidylcholine or a derivative thereof. In yet another embodiment, the cyclodextrin-lipid complex includes sphingomyelin (SM), 1-dioleoyl phosphatidylcholine (DOPC), 1-palmitoyl 2-oleoyl phosphatidylcholine (POPC), 1-stearoyl-2-oleoyl-phosphatidylcholine (SOPC) and/or combinations thereof.

In certain embodiments, the lipids incorporated in cyclodextrin-lipid complexes are extracted from the cell membrane of cells, and isolated for use in the present lipid exchange methods. For example, lipids can be removed from the plasma membrane of a first sample of cells by methods known by one of ordinary skill in the art. These lipids can then be isolated (recovered, and separated) by, for example, chromatography, e.g., thin layer chromatography or HPTLC. The isolated lipids can then be reconstituted and incubated with a cyclodextrin to form cyclodextrin-lipid complexes of the present disclosure. In certain embodiments, specific membrane lipid species can be further selected from the isolated lipids in order to facilitate the exchange of a particular type of membrane lipid and the examination of its physiological function in a cell.

In a specific embodiment, methods for extracting lipids from cells, such as a hexane-isopropanol method, a hexane-methanol based method or a chloroform-methanol based extraction method include, obtaining cells, and pelleting the cells using centrifugation, mixing cell extracts (pellet) with a hexane-isopropanol, a hexane-methanol or a chloroform-methanol extraction buffer, vortexing the mixture and incubating over time. The solution is then centrifuged to precipitate cellular debris and the organic solvent phase of the mixture, which contains the cellular lipids is collected. The lipids are then dried for further use and/or analysis by known methods such as mass spectrometry, chromatography or scintillation.

In one embodiment, the lipid of a cyclodextrin-lipid complex can be an unnatural lipid. Non-limiting examples of unnatural lipids for use in the present methods include lipids with modified acyl chain, length(s), composition, function or a combination thereof. More specifically, unnatural lipids include lipid analogs that are modified in such a manner that they are not subject to phospholipase mediated enzymatic activity. The incorporation of unnatural lipids in living cells can facilitate, for example, the study of signal transduction pathways, cellular membrane function, protein-protein interaction, and various pathologies derived therefrom. In a specific embodiment, the unnatural lipid bound to an alpha-cyclodextrin in a cyclodextrin-lipid complex of the present disclosure is N-hepadecanoyl-D-erythro-sphingosylphosphorylcholine (C_{17:0} SM).

In yet another embodiment, lipids of the present disclosure can be modified (e.g., labeled). In one embodiment, the endogenous lipids present in the cellular membrane are labeled such that the endogenous lipids can be identified or detected by any means known to one of ordinary skill in the art, e.g., detection of radioisotopes, fluorescence spectroscopy, and fluorescent microscopy, fluorescent-activated cell sorting (FACS), chromatography, such as, thin-layered chromatography (TLC), or high-performance TLC (HPTLC).

A lipid may be labeled by any means known to one of ordinary skill in the art or by using any commercially available or improvised method. Certain non-limiting examples of such labeling means include incorporating a radio isotope on a lipid (e.g., ³H, ³P), a fluorescently labeled lipid (e.g., fluorophore, fluorescent dye, fluorescent protein or quantum dots), ligand binding groups (e.g. biotin), chemical linkers and crosslinkers (e.g. sulfhydryls and alkynes), spin labeled for electron spin resonance (ESR) experiments, and lipids isotope labeled (e.g. ²H, ¹³C) for nuclear magnetic resonance (NMR) experiments. In specific embodiments, lipids can be labeled with ³H acetate and measured using a scintillation counter to measure radiation. In a, preferred embodiment, lipids can be labeled with 7-nitro-2-1,3-benzoxadiazol-4-yl (NBD) or rhodamine and measured by fluorescence spectroscopy analysis.

Formation of the cyclodextrin-lipid complexes of the present disclosure includes incubation of aqueous dispersions of lipids with cyclodextrins in solution, which enables the lipids to bind the hydrophobic cores of the cyclodextrins. Generally, the cyclodextrin-lipid complexes are formed separately, and then administered to cells.

Generally, the present methods include the following steps; an amount of lipid is dissolved in an organic solvent and dried in a vacuum environment. Next, a desired amount of a dried lipid (e.g., SM) is mixed with an amount of medium, such as RPMI 1640 medium without serum. This medium is then incubated at 70° C. to form multilamellar vesicles (MLV) containing lipids for use in the present lipid exchange methods. After the formation of MLVs containing the desired lipid, a desired amount of cyclodextrin (e.g., αCD, or MαCD) is added to the mixture containing the MLV and mixed. The cyclodextrin and MLV are then incubated together at 37° C. for about 30 minutes to form cyclodextrin-lipid complexes. Without being bound by theory, during the incubation step, lipids separate from the MLV and bind to the hydrophobic interior cavity of a cyclodextrin molecule to form a cyclodextrin-lipid complex.

As shown in Table 2, the concentration of lipids and cyclodextrin can vary based upon cell type or other experimental conditions. The appropriate concentration of lipids and cyclodextrin can be determined by one of ordinary skill in the art using known techniques without undue experimentation.

In certain embodiments, the appropriate lipid concentration can be determined prior to implementing the exchange methods of the instant disclosure. For example, the optimal lipid concentration can be determined by screening a series of various lipid concentrations. For example, cyclodextrin-lipid complexes of the present disclosure can be formed using a constant alpha-cyclodextrin concentration and varying lipid. Once various solutions of cyclodextrin and lipid are made they can be applied to cells using the methods described herein. This will enable the user to identify the highest concentration(s) lipids where the cells are not negatively affected, i.e., no cell rounding over time is optimal for lipid exchange, as it minimizes cell loss during processing. This can be determined, for example, by splitting cells into each well of a multi-well plate one day before the experiment and growing the cells to confluence. Then equal aliquots of the solutions having various concentrations of lipid are placed in separate wells and incubated at 37° C. for 1-2 hours. Cell condition (e.g., morphology, viability) is checked by microscope periodically in order to identify which concentration of lipid does not cause cell rounding during the incubation period.

In one embodiment, the lipid concentration is 12.0 mM or less. In other embodiments, the lipid concentration is less than 6.0 mM. In yet other embodiments, the lipid concentration is less than 3.0 mM. In other embodiments, the lipid concentration is less than 2.0 mM. In certain embodiments, the lipid concentration is about 1.5 mM. In other embodiments, the lipid concentration is between 0.2 mM and 12.0 mM. In yet another embodiment, the lipid concentration is between 0.5 mM and 6.0 mM. In another embodiment, the lipid concentration is between 1.0 mM and 3.0 mM. In another embodiment, the lipid concentration is between 1.0 mM and 2.0 mM. In preferred embodiments, the lipid concentration used to form cyclodextrin-lipid complexes is 0.5 mM, 1.0 mM, 1.5 mM, 2.0 mM, 2.5 mM, 3.0 mM, 5.0 mM or 6 mM. In specific embodiments, the lipid is sphingomyelin (SM), 1-dioleoyl phosphatidylcholine (DOPC), 1-palmitoyl 2-oleoyl phosphatidylcholine (POPC), or 1-stearoyl-2-oleoyl-phosphatidylcholine (SOPC) at a, concentration of about 1.5 mM. In other embodiments, the lipid is sphingomyelin (SM) at a concentration of 1.5 mM.

Also, shown in Table 2, the cyclodextrin concentration used to form cyclodextrin-lipid complexes can vary based on the type of lipid, cell type or other experimental condition.

In certain embodiments, the appropriate cyclodextrin concentration can be determined prior to implementing the exchange methods of the instant disclosure. For example, the optimal cyclodextrin concentration can be determined by screening a series of various cyclodextrin concentrations. For example, cyclodextrin-lipid complexes of the present disclosure can be formed using a constant lipid concentration but varying alpha-cyclodextrin concentration. Once various solutions of cyclodextrin and lipid are made they can be applied to cells using the methods described herein. This will enable the user to identify the highest concentration(s) cyclodextrin that does not negatively affect cells, i.e., no cell rounding over time is optimal for lipid exchange, as it minimizes cell loss during processing. This can be determined, for example, by splitting cells into each well of a multi-well plate one day before the experiment and growing the cells to confluence. Then equal aliquots of the solutions having various concentrations of cyclodextrin are placed in separate wells and incubated at 37° C. for 1-2 hours. Cell condition (e.g., morphology, viability) is checked by microscope periodically in order to identify which concentration of lipid does not cause cell rounding during the incubation period.

In certain exemplary embodiments, the cyclodextrin concentration used to form cyclodextrin-lipid complexes and is any value within the range of 0-80 mM. In one embodiment, the cyclodextrin concentration is less than 80 mM. In other embodiments, the cyclodextrin concentration is 40 mM or less. In some embodiments, the cyclodextrin concentration is between 20 mM and 80 mM. In yet another embodiment, the cyclodextrin concentration is between 40 mM and 70 mM. In other embodiments, the cyclodextrin concentration is about 40 mM. In preferred embodiments, the cyclodextrin is methyl-α-cyclodextrin at a concentration of 2 mM, 5 mM, 10 mM, 20 mM, 40 mM, or 80 mM.

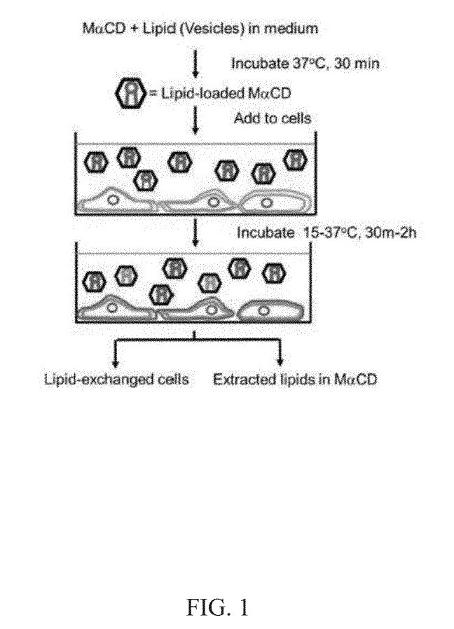

Next, as exemplified in FIG. 1 the lipid-loaded cyclodextrin-lipid complexes are formed by incubating a desired amount of α-cyclodextrin with lipid containing vesicles (MLV) for approximately 30 minutes at 37° C. However, longer or shorter incubation periods are also applicable. In a specific embodiment, an amount of MαCD, such as from a stock solution of MαCD is dissolved in DPBS and mixed with RPMI 1640 medium without serum (to give a concentration of 2-times the desired amount of MαCD). This mixture is then added to an equal volume of MLV solution, and added into a conical centrifuge tube. Then, in order to generate cyclodextrin-lipid complexes of the present disclosure, the mixture is placed in a 37° C. incubator for 30 min. The cyclodextrin-lipid complexes are then incubated with cells to facilitate the exchange of endogenous lipids in the cell membrane and exogenous lipids of the cyclodextrin-lipid complexes.

In certain embodiments, the cells are incubated with the cyclodextrin-lipid complexes for a duration of between 1 min and 20 hours. As shown in Table 2, the duration of incubation of cells with the cyclodextrin-lipid complexes of the present disclosure can vary based on the type of cell, concentration of cyclodextrin, as well as concentration and type of lipid. In specific embodiments, the incubation is for a duration of from 15 min to 6 hours. In other embodiments, the incubation is for a duration of from 30 min to 6 hours. In some embodiments, the incubation is for a duration of less than 1 hour. In other embodiments, the cells are incubated with the cyclodextrin-lipid complexes for about 1 hour, about 2 hours, about 3 hours, about 4 hours or about 6 hours. However, longer or shorter incubation periods are also applicable.

In certain specific embodiments, the temperature during incubation is between about 15° C. and about 42° C. or 15° C. and about 37° C. In other embodiments, the cells are incubated with the cyclodextrin-lipid complexes at a temperature of about 15° C. or about 37° C.,

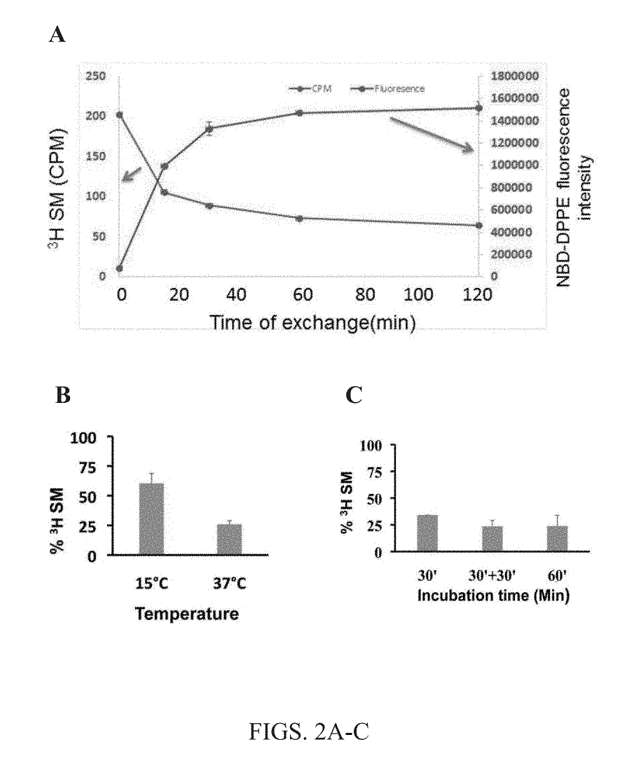

In some embodiments, the cells are incubated with the cyclodextrin-lipid complexes at a temperature of 15° C. or 37° C. For example, in the exemplary embodiment shown in FIGS. 2A-C, a 1500 μl aliquot of cyclodextrin-lipid complex composed of 1.5 mM lipid (e.g., phospholipid, sphingolipid or a combination thereof) and 40 mM methyl-αCD is added to the media in 100 cm culture dish containing 90% confluent mammalian cells (A549 cells) cultured in dishes, and incubated for about 1 hour at 37° C. to facilitate the exchange of lipids between the cyclodextrin-lipid complexes of the present disclosure and the plasma membrane of the cells.

In yet another embodiment of the present disclosure and as shown in FIG. 1, after the incubation step, an aqueous buffer is used to remove excess cyclodextrin-lipid complexes and the endogenous cell membrane lipids exchanged from the cell membrane from the cell media.

For example, in a specific embodiment, the method includes providing plates of cells grown on 10 cm plates to 90-100% confluence and removing the grown media by washing in PBS. After aspiration of the cellular media an aliquot of solution that includes the cyclodextrin-lipid complex of the present disclosure is added to the cells. The cells are then incubated in the cyclodextrin-lipid complex containing solution for 1 hour at 37° C. in a 5% CO₂ incubator with gentle rocking. At the end of the incubation the cyclodextrin-lipid complex containing solution is removed and the cells are washed in PBS. After washing, the cells can be removed from the plate and pelleted by centrifugation for further analysis of membrane lipid content or cells can be maintained in culture with new growth media.

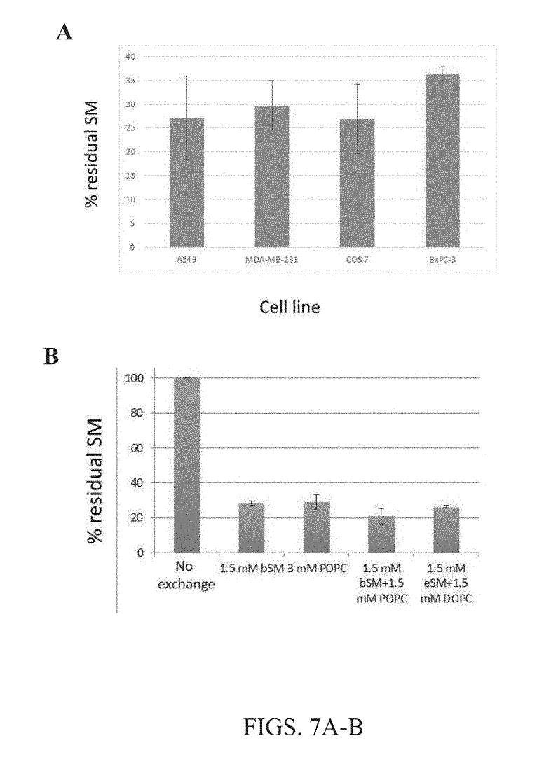

Generally, as shown in FIG. 7A any cell containing a cellular membrane can be used in the present methods. In specific embodiments, the cells have a plasma membrane composed of a lipid bilayer. In certain embodiments, the cells of the present methods are prokaryotic cells. In a preferred embodiment the cells are mammalian cells. In another embodiment, the cells are insect cells. In yet another embodiment, the cells are bacterial cells. Non-limiting examples of specific cells for use in the present methods include, kidney cells (COS-7), breast tissue (MDA-MB-231), epithelial cells (A549) or pancreatic cells (BxPC-3).

In certain embodiments, such as that exemplified in FIGS. 2A-C, prior to incubation of the cells with cyclodextrin-lipid complexes, in order to track the exchange of lipids between cyclodextrin-lipid complexes and the cells, the cells and/or cyclodextrin-lipid complexes can be incubated with a label that binds to either the endogenous lipids present in the cell membrane or exogenous lipids in the cyclodextrin-lipid complexes.

For example, cells can be incubated with ³H acetate overnight and then washed with PBS to remove excess ³H acetate. Next, a desired amount of cyclodextrin-lipid complex is added to the cell media and incubated as stated above. After incubation with cyclodextrin-lipid complex, the supernatant can be collected and analyzed by chromatography (e.g., HPLC) to detect the presence of ³H labeled lipids in the media (i.e., exchanged by the present methods).

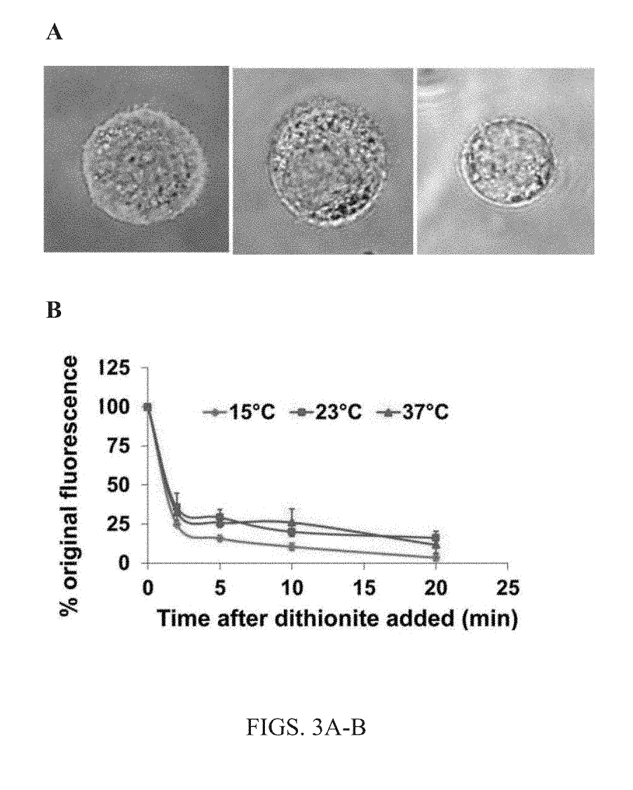

In yet another example, as shown in FIG. 3A, the cyclodextrin-lipid complexes or endogenous cellular lipids can be incubated with a fluorescent label, e.g., NBD-DPPE, to incorporate the fluorescent label. Next, a predetermined amount of labeled (or not) cyclodextrin-lipid complex is added to the cell media and incubated. After incubation with the cyclodextrin-lipid complex, the cells are collected and analyzed by fluorescent microscopy to determine whether lipids were successfully exchanged between the cellular membrane and the cyclodextrin-lipid complexes of the present disclosure. Additionally, as shown in FIG. 3B cells can be incubated with dithionite after the exchange of NBD-labeled lipids from cyclodextrin-lipid complexes and fluorescence can be analyzed by way of fluorescence spectroscopy and the amount of fluorescence detected compared before and after treatment with dithionite.

For example, the methods of the present disclosure can also include one or more methods analysis of lipid exchange. In certain embodiments, the methods of the instant disclosure include the analysis of lipid exchange including extracting lipids from cells, by known methods, such as a hexane-isopropanol method, a hexane-methanol based method or a chloroform-methanol based extraction method. Such extraction methods generally include, mixing cell extracts (pellet) with a hexane-isopropanol, a hexane-methanol method or a chloroform-methanol extraction buffer, vortexing the mixture and incubating over time. The solution is then centrifuged to precipitate cellular debris and the organic solvent phase of the mixture, which contains the lipids is collected. The lipids are then dried for further use and analysis by known methods such as mass spectrometry, chromatography or scintillation.

### Kits

Another aspect of the present disclosure includes kits containing materials and instructions for the exchange of membrane lipids in living cells. Exemplary kits of the present disclosure include a cyclodextrin-lipid complex composition of the present disclosure, and optionally contain instructions for use in conjunction with the methods of the instant disclosure. The instructions may be in any suitable format, including, but not limited to, printed matter, DVD, CD, USB or directions to internet-based instructions.

In some embodiments, the kits comprise a container with or without a label. Suitable containers include, for example, bottles, vials, and test tubes. The containers may be formed from a variety of materials such as glass or plastic. In certain embodiments the kits of the present disclosure include containers, such as 15 mL conical tubes, 50 mL conical tubes, 1.5 mL centrifuge tubes, glass tubes (e.g., 10 mL), 10 cm cell culture dishes or a combination thereof. The label on the container may indicate the contents (e.g., lipids, cyclodextrin, cells, solution, solvent, buffer) and may also indicate directions for storage, either in vivo or in vitro uses such as those described herein.

In one embodiment, a kit for substituting membrane lipids in a cell includes a container of membrane lipids such as, phospholipids and/or sphingolipids, either dried or in solution, and a container that includes an amount of cyclodextrins, such as alpha-cyclodextrin or a methyl-alpha-cyclodextrin, and instructions for use. The container may be any of those known in the art and appropriate for storage and delivery of chemicals, cells, or other biological material.

In some embodiments, kits of the present disclosure include at least one container of lipids such as, phospholipids and/or sphingolipids. The lipids provided can dried (lyophilized) or in solution. In embodiments, where the lipids are in solution they are dissolved in a solution comprising chloroform and provided in a glass container. As stated above, in certain embodiments the lipids are lipids commonly found in the cell membrane such as, for example, lipids of the outer leaflet of the plasma membrane. For example, any lipid that includes a polar head group and acyl chain(s) can be used to form cyclodextrin-lipid complexes of the present disclosure. In specific embodiments, the lipids are exogenous phospholipids or sphingolipids. In preferred embodiments of the present disclosure, the sphingolipid is a sphingomyelin (SM) or a derivative thereof. In specific embodiments of the present disclosure, the phospholipids is phosphatidylcholine (PC) or a derivative thereof.

In some embodiments, the lipids are brain sphingomyelin (bSM), egg sphingomyelin (eSM), milk sphingomyelin (mSM), 1-palmitoyl-2-oleoyl-sn-glycerol-3-phosphocholine (POPC), 1,2-dioleoyl-sn-glycero-3-phosphocholine (DOPC), phosphatidylethanolamine (PE), phosphatidylserine (PS), 1-palmitoyl-2-oleoyl-sn-glycero-3-phosphoethanolamine (POPE), 1-stearoyl-2-oleoyl-phosphatidylcholine (SOPC) and -palmitoyl-2-oleoyl-sn-glycero-3-phospho-L-serine (POPS), 1,2-dipalmitoyl-sn-glycero-3-phosphoethanolamine-N-(7-nitro-2-1,3-benzoxadiazol-4-yl) (NBD-DPPE), NBD-SM or NBD-POPC.

In certain embodiments, the lipids incorporated in kits of the present disclosure are endogenous cell membrane lipids (SM, PC, PE, PS), which have been extracted from the plasma membrane of cells, and isolated. In yet another embodiment, lipids are modified (e.g., labeled). For example, labeled lipids are radio-labeled lipids or fluorescent dye-labeled lipids. Radiolabeled lipids of the present disclosure include lipids that incorporate ³H or ¹⁴C isotopes, such as ³H-SM, or ¹⁴C-SM. Fluorescently labeled lipids of the present disclosure can include lipids labeled with NBD, such as 1,2-dipalmitoyl-sn-glycero-3-phosphoethanolamine-N-(7-nitro-2-1,3-benzoxadiazol-4-yl) (NBD-DPPE), NBD-SM and NBD-POPC. In some embodiments, the lipids included in kits are unnatural lipids. In a specific embodiment, the unnatural lipid has a non-naturally occurring acyl group, such as C^{17:0} SM.

In some embodiments, the lipids included in kits of the present disclosure are included in multilamellar vesicles (MLV). For example, a desired amount of a dried lipid (e.g., SM) is included in a container including an amount of medium, such as RPMI 1640 medium without serum.

In specific embodiments, the kits of the present disclosure include a methyl-α-cyclodextrin solid or dissolved in water or PBS (e.g., DPBS (GIBCO™) to make a stock solution of a methyl-α-cyclodextrin. The concentration can be any desired concentration, such as about 400 mM a methyl-α-cyclodextrin solution. In certain embodiments, the stock solution of a methyl-α-cyclodextrin is between 300 mM and 400 mM MαCD in DPBS. In a specific embodiment, the stock solution of a methyl-α-cyclodextrin is about 380 mM MαCD in DPBS. In other embodiments, the stock solution of a methyl-α-cyclodextrin is between 300 mM and 400 mM MαCD in water. In a specific embodiment, the stock solution of a methyl-α-cyclodextrin is about 380 mM MαCD in water.

In certain embodiments, the kit further comprises a third container comprising cells for preparation use in any of the above methods. The cells can be cryogenically frozen, once those cells have been unfrozen, or live cells. In specific embodiments, the cells have a plasma membrane composed of a lipid bilayer. In certain embodiments, the cells are prokaryotic cells. In a, preferred embodiment, the cells are mamnmalian cells. In another embodiment, the cells are insect cells. In yet another embodiment, the cells are bacterial cells. Non-limiting examples of cells for incorporation in kits of the present disclosure include, kidney cells (COS-7), breast tissue (MDA-MB-231), epithelial cells (A549) or pancreatic cells (BxPC-3).

In other embodiments, the kits of the present disclosure may further include other materials desirable from a commercial and user standpoint including, but not limited to, buffers, diluents, media, culture dishes, test tubes, antibodies, dyes, chemicals, filters, needles, syringes, and package inserts with instructions for performing any methods described herein.

In some embodiments, the kits of the present disclosure include medium. In some embodiments, the medium included in a kit is RPMI 1640 medium with serum or without serum. In certain embodiments, a kit includes a container of RPMI 1640 medium with serum and a container of RPMI 1640 medium without serum. However, other types of medium known by one of ordinary skill in the art are also contemplated.

In certain embodiments, the kits of the present disclosure include at least one buffer. In some embodiments, the buffer is an extraction buffer for the extraction of lipids from cells. In specific embodiments the extraction buffer includes hexane:isopropanol or hexane:ethanol. In certain embodiments, the extraction buffer includes 3:2 (v:v) hexane:isopropanol. In some embodiments the extraction buffer includes chloroform and methanol. In some embodiments the buffer provided in a kit of the instant disclosure includes PBS, such as DPBS.

Kits of the present disclosure also include package inserts with instructions for performing methods of using the compositions of the present disclosure. In certain embodiments, the package inserts include instructions that enable the formation of a cyclodextrin-lipid complex composition of the present disclosure. In some embodiments, the package insert includes instructions that enable the use of a cyclodextrin-lipid complex composition of the present disclosure to exchange membrane lipids with a cell. In some embodiments, the instructions include methods for the preparation of a, stock solution of a component of the kit, such as a stock solution of alpha-cyclodextrin in water or PBS, a stock solution of a lipid in a solvent such as chloroform or both, as well as methods for measuring the concentration of a component of such stock solution(s).

Instructions in a kit of the present disclosure include methods for the preparation of a stock solution of methyl-alpha-cyclodextrin and confirming the concentration of MαCD in the solution. For example, the instructions include the following directions MαCD solid (AraChem, The Netherlands, CDexA-066) is dissolved in water or in DPBS (GIBCO, 14190-144). Specifically, 6.4 g MαCD dissolved in 12 ml DPBS to make a, roughly 380 mM MαCD solution. The MαCD solution can then be filtered using a BD 10 mL syringe equipped with a 0.2-μm pore filter. The final stock solution concentration in water can be measured directly by dry weight by cutting aluminum foil into squares, numbered on the exterior surface, and then shaped into a liquid-tight container. Then, using forceps, the foil container is weighed to the closest microgram, and the weight is recorded. Next, 10 μl of MαCD solution is added into each container. The solution in each container is then dried under a gentle stream of nitrogen. The containers are then placed in a, high vacuum to remove residual water. It is essential that drying is complete. Each container is then reweighed, and the difference in weight is used to calculate the concentration of the stock solution. For example, for MαCD solution in DPBS final concentration is measured by comparison to a standard curve. The index of refraction of MαCD/DPBS can be measured using a refractometer and compared to that for a standard curve of MαCD calibrated by dry weight.

Instructions in a kit of the present disclosure include methods for the preparation of a stock solution of lipids and confirming the concentration of lipids in the solution. For example, the instructions include the following directions lipids are provide in a lyophilized solid form or dissolved in chloroform (Avanti, 860062C or 860062P). If solid, the lipid is dissolved in chloroform and stored in glass at −20° C. or −70° C. until use. The final stock lipid solution concentration can be measured directly by dry weight by cutting aluminum foil into squares, numbered on the exterior surface, and then shaped into a liquid-tight container. Then, using forceps, the foil container is weighed to the closest microgram, and the weight is recorded. Next, the lipid/chloroform solution is warmed to dissolve any lipid precipitate and a pre-determined amount (e.g., 10 μl) is added into each container. The solution in each container is then dried under a gentle stream of nitrogen. The containers are then placed in a high vacuum to remove residual water. It is essential that drying is complete. Each container is then reweighed, and the difference in weight is used to calculate the concentration of the stock solution.

Instructions in a kit of the present disclosure include methods for the preparation of multilamellar vesicles (MLV) including lipids for the formation of the cyclodextrin-lipid complexes of the present disclosure. For example, in certain embodiments the instructions include providing an amount of lipid solution (e.g., from a lipid stock solution) and depositing the amount of lipid solution into a glass tube (e.g. VWR, 47729). The lipid solution is then dried under a gentle stream of nitrogen until no liquid is detected. It is essential that drying is complete. Concurrently to the drying step, RPMI 1640 medium without serum is being warmed in a conical tube. Next, a volume of RPMI 1640 medium without serum is added into the glass tube containing the dried lipids such that a lipid concentration that is 2-times the desired concentration (i.e. 3 mM when the cells are to be incubated with 1.5 mM lipid) if provided. The solution is then vortexed. The glass tube is sealed and incubated in a 70° C. water bath for 5 min, vortexing every minute, which forms lipid containing MLVs of the present disclosure.

Instructions in a kit of the present disclosure can include methods for the preparation of a cyclodextrin-lipid complex of the present disclosure. For example, in certain embodiments, the instructions include providing an amount of MαCD from a stock solution of MαCD dissolved in DPBS and mixing the MαCD with RPMI 1640 medium without serum (to give a concentration of 2-times the desired amount of MαCD). This mixture is then added to an equal volume of MLV solution, prepared as described above, and added into a conical centrifuge tube. The mixture is vortexed briefly. To generate cyclodextrin-lipid complexes of the present disclosure, the mixture is placed in a 37° C. incubator for 30 min.

Instructions in a kit of the present disclosure include methods for the exchange of lipids between cells and cyclodextrin-lipid complexes of the present disclosure. For example, in certain embodiments, the instructions include providing plates of cells grown on 10 cm plates to 90-100% confluence and removing the grown media by washing in PBS. After aspiration of the cellular media an aliquot of solution that includes the cyclodextrin-lipid complex of the present disclosure is added to the cells. The cells are then incubated in the cyclodextrin-lipid complex containing solution for 1 hour at 37° C. in a 5% CO₂ incubator with gentle rocking. At the end of the incubation the cyclodextrin-lipid complex containing solution is removed and the cells are washed in PBS. After washing, the cells can be removed from the plate and pelleted by centrifugation for further analysis of membrane lipid content or cells can be maintained in culture with new growth media.

Instructions in a kit of the present disclosure can also include one or more methods analysis of lipid exchange. In certain embodiments, the instructions for the analysis of lipid exchange include methods for extracting lipids from cells, such as a, hexane-isopropanol method, a hexane-methanol based method or a chloroform-methanol based extraction method. Such extraction methods are known to one of ordinary skill in the art to generally include, mixing cell extracts (pellet) with a hexane-isopropanol, a hexane-methanol method or a chloroform-methanol extraction buffer, vortexing the mixture and incubating over time. The solution is then centrifuged to precipitate cellular debris and the organic solvent phase of the mixture, which contains the lipids is collected. The lipids are then dried for further use and analysis by known methods such as mass spectrometry, chromatography or scintillation.

In some embodiments, the instructions for the analysis of lipid exchange include methods for identifying optimal concentrations of lipids and/or cyclodextrins for use in exchanging lipids between a cyclodextrin-lipid complex of the present disclosure and a cell membrane. Since cells are sensitive to the amount of alpha-cyclodextrin and lipid provided (see Table 2), it is important to identify optimal cyclodextrin and lipid concentrations before carrying out lipid exchange in live cells. As such, instructions included within kits of the instant disclosure can include, instructions for screening a series of various cyclodextrin or lipid concentrations. For example, the instructions can include maintaining a constant lipid concentration and altering numerous alpha-cyclodextrin concentrations or vice versa. Once various solutions of cyclodextrin and lipid are made they can be applied to cells using the methods described herein. This will enable the user to identify the highest concentration(s) where the cells are not negatively affected, i.e., no cell rounding over time is optimal for lipid exchange, as it minimizes cell loss during processing. This can be determined, for example, by splitting cells into each well of a multi-well plate one day before the experiment and growing the cells to confluency. Then equal aliquots of the solutions having various concentrations are placed in separate wells and incubated at 37° ° C. for 1-2 hours. Cell condition (e.g., morphology, viability) is checked by microscope periodically in order to identify which concentration of mixture does not cause cell rounding during the incubation period.

The methods, compositions and kits of the present disclosure will be better understood by reference to the following examples, which are provided as exemplary of the disclosure and not by way of limitation.

### EXAMPLES

### Example 1: Materials and Methods

Materials. Breast cancer cell line MDA-MB-231, COS 7 kidney cells, A549 lung carcinoma and BxPC-3 pancreatic cells are from ATCC. Brain sphingomyelin (bSM), egg sphingomyelin (eSM), milk sphingomyelin (mSM), N-hepadecanoyl-D-erythro-sphingosylphosphorylcholine (C_{17:0} SM), 1-palmitoyl-2-oleoyl-sn-glycerol-3-phosphocholine (POPC), 1,2-dioleoyl-sn-glycero-3-phosphocholine (DOPC), and 1,2-dipalmitoyl-sn-glycero-3-phosphoethanolamine-N-(7-nitro-2-1,3-benzoxadiazol-4-yl) (NBD-DPPE), 1-palmitoyl-2-oleoyl-sn-glycero-3-phosphoethanolamine (POPE), 1-palmitoyl-2-oleoyl-sn-glycero-3-phospho-L-serine (POPS), and cholesterol were from Avanti Polar Lipids (Alabaster, Ala.). ³H acetate was from PerkinElmer, Inc. (Boston, Mass.). Methyl-α-cyclodextrin (MαCD) was purchased from AraChem (Budel, the Netherlands). Methyl-β-cyclodextrin (MPCD), FITC-conjugated cholera toxin B (CTxB) and paraformaldehyde (PFA) powder were purchased from Sigma Aldrich (St. Louis, Mo.). RPMI medium 1640, DMEM medium, fetal bovine serum, Hank's balanced salt solution (HBSS), Dulbecco's phosphate-buffered saline, 200 mg/L KCl, 200 mg/L KH₂PO₄, 8 g/L NaCl, and 2.16 g/L Na₂H PO₄ (DPBS) were Gibco brand and purchased from Life Technologies (Grand Island, N.Y.). Transferrin conjugated with Alexa Fluor® 488 (TF-AF488) and CellMask™ Deep Red (plasma membrane staining solution) were Molecular Probes brand and obtained from Life Technologies (Eugene, Oreg.). VECTASHIELD® mounting medium was bought from Vector Laboratories, Inc. (Burlingame, Calif.). Bovine serum albumin (BSA) was obtained from Millipore (Kankakee, Ill.). 10× phosphate buffered saline (PBS, pH 7.8±0.2) was bought from Bio-Rad Laboratories Inc. (Hercules, Calif.) Citric acid and sodium chloride were purchased through Fisher Scientific (Fair Lawn, N.J.). Distilled and de-ionized water was used. Other chemicals were reagent graded.

Cell Culture. A549 and BxPC-3 cells were cultured in RPMI medium 1640, or DMEM medium depending upon cell type, to facilitate growth and cell maintenance. DMEM medium was used to culture MDA-MB-231 and COS 7 cells. All cell media was supplemented with 10% fetal bovine serum and all cells were cultured in an incubator at 37° C. with 5% CO₂.

High Performance Thin Layer Chromatography. The samples and pure lipid standards were dissolved in 1:1 (v:v) chloroform/methanol. In some cases lipids from the asymmetric vesicle samples were first extracted using a 2:2:1 (v:v) chloroform/methanol/water and dried under a nitrogen stream, but this did not affect the results. The dissolved lipids then applied to HP-TLC (Silica Gel 60) plates (Merck, KGaA, Darmstadt, Germany) or Uniplate Silica Gel G plates (Analtech, Inc., Newark, Del.) and chromatographed in 65:25:5 (v:v) chloroform/methanol/28.0-30.0 (v/v) % ammonium hydroxide. Following chromatography, the TLC plates were air-dried and saturated with 3% (w/v) cupric-acetate-8% (v/v) phosphoric acid by spraying, and then air-dried again. Plates were then charred at 180° C. to develop lipid bands. Lipid band intensity was measured using Image J software (National Institutes of Health). Lipids in samples were quantified by comparing background-subtracted band intensity with that of a curve composed of various standard amounts of each lipid chromatographed on the same TLC plate. The intensity in the standard bands was fit to a linear intensity versus lipid quantity curve.

Preparation of Cyclodextrin-lipid Complexes. Cyclodextrin stock solutions were prepared in 1×PBS. To prepare multilamellar vesicles, lipids were dried under nitrogen followed by 1 h vacuum. Then 2.5 mL of IX PBS pre-warmed to 70° C. was added. After 5 min incubation at 70° C., vesicles were divided into eight 250 μL aliquots. Then different volumes of the CD stock solution were added to prepare samples over a range of CD concentrations. Final samples had IX PBS and a, volume of 500 μL. Final lipid concentrations were 1.5 mM for POPC, bSM, and POPS, or 1.5 mM for 1:1 (mol:mol) POPE/POPS. After the samples were incubated for 30 min at room temperature or 37° C., they were placed in quartz cuvettes, and light scattering (optical density) measurements were carried out at a wavelength of 300 nm using a Beckman 640 spectrophotometer (Beckman Coulter, Fullerton, Calif.). IX PBS was used as the background sample.

A desired amount of lipid was dissolved in organic solvent and introduced into disposable glass tubes. Lipids were then dried by nitrogen and subjected to a vacuum environment for 1 hour. Multilamellar vesicles (MLV) composed of lipids were prepared using the dried lipids at 70° C. by adding pre-warmed RPMI 1640 medium or DMEM medium depending upon cell type. The desired amount of cyclodextrin (i.e., αCD, MαCD) was created from a 400 mM stock solution of CD dissolved in Dulbecco's Phosphate-Buffered Saline (PBS) and was added to the MLV and RPMI or MLV and DMEM mixture. The concentration of lipids and CD) used to prepare cyclodextrin-lipid complexes was dependent upon experimental requirements and objectives. However, generally, the lipid concentration was 1.5 mM and CD concentration was 40 mM. The mixture of CD and lipid was then incubated at 37° C. for 30 minutes to load the lipids onto the CD and form cyclodextrin-lipid complexes for exchange of lipids into cells.

Exchange of Lipids between Cells and Cyclodextrin-lipid Complexes. Unless otherwise noted, the cells are cultured in 10 cm plates with 10 mL medium. After removing the growth medium and washing three times with 10 ml DPBS, a 1500 μL aliquot of cyclodextrin-lipid complex containing MαCD (formed using 1.5 mM lipid and 40 mM MαCD concentrations) was added to 90% confluent mammalian cells cultured in 100 cm cell culture dishes (Corning Incorporated, Durham, N.C.). The cells were then incubated with the cyclodextrin-lipid complex for 1 hour at 37° C., unless otherwise noted. After removal of the cyclodextrin-lipid complex and three washes with 10 mL PBS the cells were removed from the plate by scraping in 5 mL PBS. The cells were then pelleted by centrifugation for 3 minutes at 3,000 rpm and the lipid composition of the pellet was analyzed. For embodiments in which there are two rounds of exchange, after removing the supernatant from the first round of exchange, fresh cyclodextrin-lipid complex solution was added to the cells and a second round of lipid exchange was carried out according to the above methods.

³H Labeling Cells, Lipid Exchange and Solvent Extraction of Lipids from Cells. Unless otherwise noted, 10 cm cell culture dishes with 70% confluent A549 cells were used. Here, 10 mL RPMI 1640 medium supplemented with 10% fetal bovine serum (FBS), 2 mM sodium acetate and 10 μCi ³H acetate was used. Cells were incubated for 24 hours and the medium was removed. The cells were then washed at least three times with 10 mL PBS supplemented with 2 mM sodium acetate. Next, 1.5 ml cyclodextrin-lipid complex solution was added to a plate. As a control, 1.5 ml of 1.5 mM bSM MLV was added to another plate without cyclodextrin. The cells were incubated at 37° C. for 1 hour. Then the supernatant mixture was removed for analysis of ³H-labelled lipids extracted from cells as described herein, and the cells were washed three times with PBS supplemented with 2 mM sodium acetate. The cells were scraped in 5 ml PBS supplemented with 2 mM sodium acetate, and pelleted by centrifugation for 3 minutes at 3,000 rpm. Lipids were extracted in 3 ml hexene:isopropanol 3:2 (v/v) and then dried with nitrogen gas while gently warming.

Separation and Analysis of ³H-Labeled Lipids. Lipids, such as isolated, labeled or unnatural lipids of the present disclosure are labeled with ³H as stated above. The lipids are then dried and dissolved in 100 μl chloroform:methanol 1:1 (v/v) and 10 μl aliquots were loaded onto a HP-TLC silica gel (Merck, KGaA, Darmstadt, Germany). The lipids were separated in chloroform:methanol:ammonium hydroxide 65:25:5 (v/v), and dried. The isolated lipids were then stained in an iodine tank and lipid bands were marked on the gel. The silica gel corresponding to specific lipid bands, identified by their position relative to lipid standards, was scraped from the plate. After addition of 2.5 ml ScintiVerse™ BD cocktail (Fisher Scintific) radioactivity was counted using a Beckman LS6500 scintillation counter (Beckman Coulter, Inc., Fullerton, Calif.).

Analysis of ³H Labeled lipids extracted from cells by cyclodextrin. The supernatant mixture from lipid exchange experiments, which contained the lipids extracted from the cell by cyclodextrin-catalyzed exchange, were collected and centrifuged at 72,000 rpm (TLA 100.3 rotor) for 30 minutes in an Optima™ TL Ultracentrifuge (Beckman) to remove cellular debris. A volume of chloroform equal to that of the supernatant was added to extract the lipids from the mixture. The extracted lipids were dried and dissolved in 100 μl methanol:chloroform 1:1 (v/v). In certain cases, due to low amounts of lipid in the supernatant extracts, 25 μg of each of non-radioactive PS, PI, SM, PC and PE were added into the lipid samples before loading onto the HPLC silica gel plate. This allowed visualization of lipid positions upon iodine staining. The separation and analysis of ³H labeled lipids were carried out as described above.

Microscopy and Kinetics of Exchange Monitored with Fluorescent Probes and ³H Acetate: Cyclodextrin-lipid complex containing 1.5 mM lipid (90% bSM and 10% NBD-DPPE) and 40 mM MαCD was prepared. Then, 500 μl of the cyclodextrin-lipid complex was added to 90% confluent A549 cells cultured in 6-well plates (Corning Inc., Durham, N.C.). The cells were incubated for different times at 37° C., and then the cyclodextrin-lipid complex was removed. After three washes with 2 ml PBS the cells were removed from the plate by scraping in 1 ml PBS. The cells were pelleted by centrifugation at 3,000 rpm for 3 minutes and re-suspended in 1 ml PBS. Seven μl aliquots of cells were loaded on microscope slides and covered with a, coverslip. NBD fluorescence was then imaged by confocal laser scanning microscopy using a Zeiss LSM 5 Pascal confocal laser scanning microscope system (Carl Zeiss AG, German).

In other experiments, fluorescence intensity was measured for the each well of cells whereby cells were placed in fluorescence cuvettes using Fluorolog (Jobin Yvon Horiba, Edison, N.J.) with an excitation wavelength of 465 nm and emission wavelength of 534 nm. A non-specific lipid transfer control was prepared in a similar fashion without cyclodextrin.

In an analogous experiment, A549 cells were ³H labeled and subjected to lipid exchange with cyclodextrin-lipid complex. The cells were collected at different times and lipids extracted and separated on TLC as described above. Radioactivity in the PS+PI and SM bands was subsequently measured by scintillation counting.

Dithionite Quenching of the NBD-DPPE Exchanged into the Cells. Cells into which NBD-DPPE was exchanged were prepared as described above, except at either 15° C., room temperature (23° C.) or 37° C. Fluorescence was measured before and (as a function of time) after the addition of a freshly prepared solution of IM dithionite in IM Tris buffer (pH 10) to the cells providing a final dithionite concentration of 50 mM.

The Effect of Methyl αCD Concentration on SM Exchange Efficiency. After ³H labeling, A549 cells were incubated with cyclodextrin-lipid complex containing 1.5 mM bSM and MαCD at concentrations of 0, 2, 5, 10, 20, 40, or 80 mM. Cells were collected and lipids were analyzed as above.

The Effect of bSM Concentration on SM Exchange Efficiency. After ³H labeling, A549 cells were treated with cyclodextrin-lipid complex containing 40 mM MαCD loaded with 0, 0.1, 0.2, 0.5, 1, 1.5, 2 or 3 mM bSM. Cells were collected and lipids were analyzed as above.

SM Exchange in Different Cell Lines. A549, COS 7, MDA-MB-231 and BxPC-3 cells grown to 70% confluence were ³H labeled as described above. Lipid exchange was carried out with cyclodextrin-lipid complex containing 1.5 mM bSM and 40 mM MαCD and lipids were analyzed as above.

SM Exchange with Different Lipid Combinations. cyclodextrin-lipid complex containing 40 mM MαCD and MLVs containing 1.5 mM bSM alone, 3 mM POPC alone, 3 mM 1:1 bSM:POPC or 3 mM 1:1 eSM:DOPC. The cyclodextrin-lipid complexes were then added to the ³H-labeled A549 cells and, after lipid extraction and TLC, ³H-SM exchange efficiency was calculated.

Analysis of exogenous lipid exchange over time after delivery into A549 cells. After a 1 h at 37° C. treatment with 40 mM MαCD mixed with 1.5 mM bSM, A549 cells were washed three times with DPBS. Then the cells were cultured for 0, 2, 4, and 6 hours in 10 mL RPMI 1640 medium supplemented with 10% fetal bovine serum. After the cells were harvested, their lipids were extracted and separated on TLC plates as described above. After charring the TLC plates as described above ImageJ software was used to analyze the intensities of the SM and PS+PI bands. The ratio SM/(PS+PI)=intensity of SM band/intensity of PS+PI band.

Measurement of Transferrin (TF) Endocytosis. MDA-MB-231 cells were plated in 2 ml RPMI 1640 at 6×10⁵ on a coverslip in a 35 mm dish. After 1 d, they were subjected to cholesterol depletion or phospholipid exchange. For cholesterol depletion, cells were washed with 1 ml DPBS (2-3 times) and treated with 10 mM M MβCD/DMEM for 30 min at 37° C. in the CO₂ incubator. For phospholipid substitution, the DPBS washed cells were treated with mixture of 40 mM MαCD and a combination of 0.75 mM bSM and 0.75 mM POPC in serum free medium for 30 rain, again while maintained at 37° C. in the CO₂ incubator. After cholesterol depletion or phospholipid exchange, cells were washed with 1 ml DPBS three times. Then 50 μg/ml TF-AF488 in DMEM was added to the cells and incubated for 10 min in room temperature in the dark. Next, cells were washed with 2 ml DPBS three times and subjected to an additional 3 washes on ice with an acid solution (100 mM citric acid, 140 mM NaCl in distilled water pH adjusted to 1.75), incubating the cells with the solution for 3 min during each wash. The acid wash solution was removed by washing three times with 2 ml of 1×PBS and the cells then fixed using 2 ml 3% PFA for 30 min (10 min on ice and 20 min at room temperature). The fixed cells were then washed with 2 ml 1×PBS three times, and cell membranes stained with 4 μl/ml of CellMask in 3% BSA/HBSS for 2-5 minutes at room temperature. After washing the cells with 2 ml 1×PBS, the coverslips were mounted on slides using VECTASHIELD® mounting medium.

Comparison of the ratio of SM/(PS+PI) before and after SM being delivery into A549 cells. 40 mM MαCD was pre-incubated with 3 mM 1:1 (mol:mol) bSM:POPC and 0.5×10⁶ cpm ¹⁴C-SM. A549 cells cultured in 35 mm plates were incubated with the mixture for 1 h at 37° C. The cells were washed 4 times with DPBS. The cells were subjected to a second round of lipid exchange either right after the initial exchange or after an additional 1 h incubation in RPMI 1640 media. The second lipid exchange was carried out with 40 mM MαCD pre-incubated with 3 mM 1:1 (mol:mol) bSM:POPC. As background in the first round of exchange, the cells were treated with 3 mM 1:1 (mol:mol) bSM:POPC and 0.5×10⁶ cpm ¹⁴C-SM without MαCD for 1 h at 37° C. As a control for no exchange in the second round of exchange cells were treated with 3 mM 1:1 (mol:mol) bSM:POPC without ML CD. The lipids were extracted from cells in 2 mL 3:2 (v:v) hexane:iso-propanol. Then radioactivity was measured in 200 μL aliquots from each sample by scintillation counting.

Phospholipid Content by LCMS/M S: Phospholipids were extracted from samples as set forth above. Extracts were diluted with internal standards (Avanti Polar Lipids) respective to the structure of phospholipid classes. The samples were prepared in a silanized 500 μL injection insert and vials for LC/MS/MS analysis. Each sample extract was assayed on a Waters' Acquity ultra-performance liquid chromatograph (Waters Corporation, Milford, Mass., USA)/AB Sciex 5500 mass spectrometry system (Framingham Mass., USA). The class specific phospholipid extracts for each sample were injected on an Agilent (Santa Clara Calif., USA) Eclipse XDB-C8 reversed phase column (4.6×50 mm, 1.8 μm particle size) for separation of molecular species within each class by gradient elution and detected by mass spectrometry utilizing scheduled multiple reaction monitoring. In this manner, a peak with unique column elution time and mass-to-fragment profile was measured. The peaks of phospholipid species were integrated and normalized to the internal standard compounds within respective classes. This provided quantification using the compound/internal standard area ratio multiplied by the internal standard concentration(s) initially added to the sample extraction.

### Example 2: Exchange of Lipid Between Cyclodextrin-Lipid Complexes and Cells

FIG. 1 of the present disclosure shows a schematic cartoon of the present cyclodextrin-lipid complex mediated exchange procedure. First, lipid in the form of multilamellar lipid vesicles (MLVs) are mixed with an α-cyclodextrin (MαCD) in order to form cyclodextrin-lipid complexes. Under the conditions recited herein, the MLV are fully dissolved by MαCD as confirmed by an observed a loss of light scattering upon incubation of the lipids with MαCD. The cyclodextrin-lipid complexes are then incubated with the cells. Lipids in the outer leaflet of the cellular membrane of the cells (endogenous lipids) exchange with the lipids loaded bound to the cyclodextrin-lipid complexes. Notably, when the cyclodextrin-lipid complexes are in excess, the entire population of endogenous phospholipids and sphingolipids in the cell membrane outer leaflet can be replaced by those bound to the cyclodextrin-lipid complexes. The lipids in the cytosolic (inner) leaflet of the plasma membrane and those in internal vacuoles will not exchange.

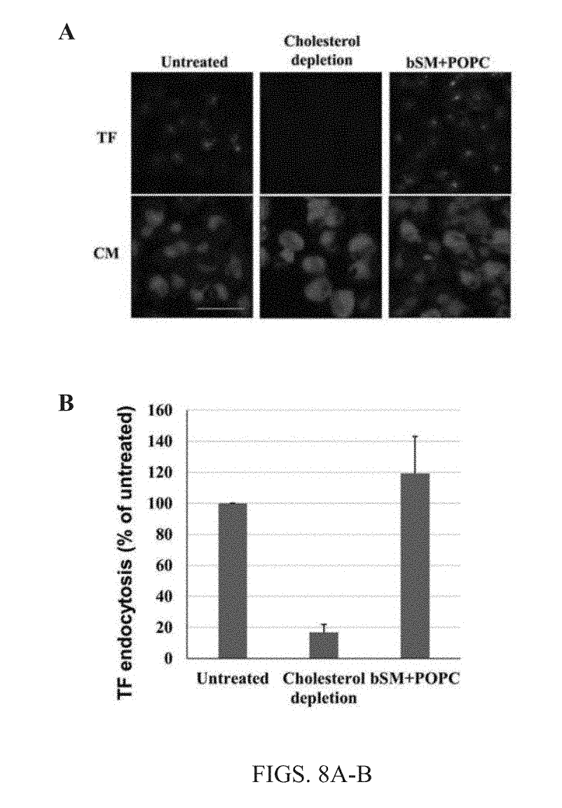

Cells were sensitive to treatment with MαCD alone, rounding up within 15 min, as shown in Table 2. To avoid this, MαCD was pre-incubated with lipid vesicles before being added to cells. After incubation of cells with the cyclodextrin-lipid complex containing solution, the solution containing MαCD and exchanged cellular lipids was removed. As shown in Table 2, incubating cells with MαCD pre-mixed with sphingolipid or phospholipid maintained normal cell morphology for up to and beyond 6 hours. After exchange under optimal conditions <1% of cells stained with trypan blue in the cytoplasm, i.e. >99% of cells survived. Maintenance of normal cell behavior after incubation with cyclodextrin-lipid complex containing mixtures was also seen by measuring clathrin-mediated endocytosis of transferrin. As shown in FIGS. 8A-B, normal endocytosis levels were observed after incubation of cells with brain SM (bSM)/POPC-loaded cyclodextrin-lipid complexes. In contrast, endocytosis was strongly inhibited after partial extraction of cellular cholesterol with MβCD.

### Example 3: Lipid Exchange and Exchange Kinetics

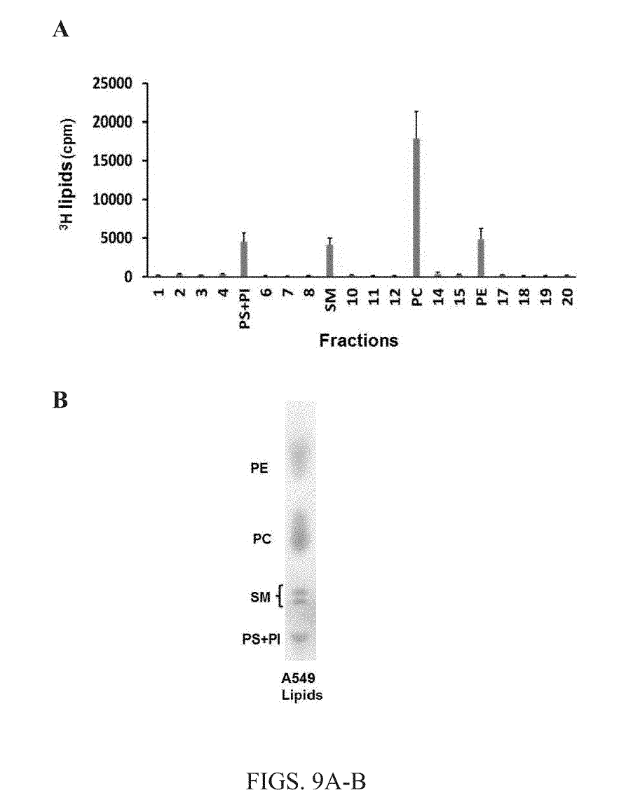

The amount of lipid exchanged into and out of cells was examined by TLC and/or fluorescent detection methods, whereby the exchange of ³H labeled cellular lipids or the exogenously added fluorescently labeled lipid NBD-DPPE were tracked. Endogenous cellular lipids were metabolically-labeled with ³H acetate, and cellular lipids before and after exchange were then quantified by extraction followed by TLC and measurement of radioactivity in each lipid band. FIG. 9A shows the predominant cell phospholipids were PC, SM, PS+PI, and PE, and Table 1 shows that the relative level of radio-labeling for each lipid was roughly consistent with their bulk concentration in cells as assessed by charring of HP-TLC plates (FIG. 9B), and mass spectrometry (Table 1).

To induce lipid exchange, exogenous lipids were mixed with αCD (MαCD) to form cyclodextrin-lipid complexes, these cyclodextrin-lipid complexes were then added to cells grown on cell culture plates. After incubation and washing, the cells were removed then fluorescence and/or radioactivity were measured. As shown in FIG. 2A, both αCD-dependent introduction of NBD-DPPE into cells and the loss of radiolabeled endogenous lipid (SM) out of cells was seen. Both lipid introduction and loss exhibited similar kinetics, consistent with an exchange process.

Exchange was determined by monitoring the introduction of exogenous fluorescently-labeled lipid NBD-DPPE into cells, and from measuring the extent of MαCD-catalyzed removal of cellular lipid. Lipid levels after exchange were then compared to those before exchange to determine efficiency. As shown in FIG. 2A, delivery of NBD-DPPE and removal of endogenous SM, a lipid predominantly located in PM outer leaflets, followed similar kinetics, with a half-time on the order of 15-20 min at 37° C. FIGS. 2B and 2C clearly show that exchange at 15° C. was about two-fold less after 1 h (FIG. 2B) yet was generally unaltered at constant temperature at different incubation times (30 or 60 minutes) or with two consecutive (30 minute) exchange steps (FIG. 2C). Association of exogenous NBD-DPPE with cells in the absence of MαCD was negligible, showing that MαCD was necessary for lipid exchange, as experiments show very little or no delivery of exogenous bSM in the absence of MαCD. The observation that the kinetics of lipid delivery and removal in cells mirrored each other is consistent with 1:1 lipid exchange. For example, the amount of fluorescent lipid (exogenous lipid) associated with cells in the absence of MαCD was only about 1/100th of the amount of fluorescent lipid associated with cells in the presence of αCD, indicating that non-specific association of lipids with cells was negligible. Notably, the possibility that the effect of MαCD mediated exchange was due to an increase in carbohydrate-mediated non-specific attachment of exogenous lipids to cells was ruled out by experiments in which 40 or 240 mM sucrose was substituted for MαCD. Here, the presence of sucrose did not increase the amount of fluorescent lipid delivered to cells relative to those cells incubated with buffer only.

As shown in FIG. 3A, confocal fluorescent microscopy of a cross-sectional slice reveals that the NBD-DPPE (exogenous lipid) loaded cyclodextrin-lipid complexes delivered exogenous lipids to the plasma membrane. This exchange is exemplified by the accessibility of the cell-associated NBD-DPPE to reduction by the membrane-impermeable reagent dithionite. For example, NBD-DPPE must be associated with the outer leaflet of the cellular membrane in order to be subject to dithionite reduction because lipid translocated to the inner leaflet or cytosol is protected from dithionite reduction. Notably, the trace amount of fluorescence seen in the cell interior after reduction by dithionite is consistent with entry of NBD-DPPE into internal vacuoles by endocytosis. These results were confirmed by the measurement of bulk NBD fluorescence upon treatment with dithionite, as shown in FIG. 3B. For example, upon incubation of cells with cyclodextrin-lipid complex at 37° C. approximately 30% of the fluorescence was protected from dithionite reduction (extrapolating to time zero to correct for slow dithionite permeation into cells). However, protection decreased to about 20% when incubation was carried out at 15° C., because endocytosis is prohibited at the lower temperatures.

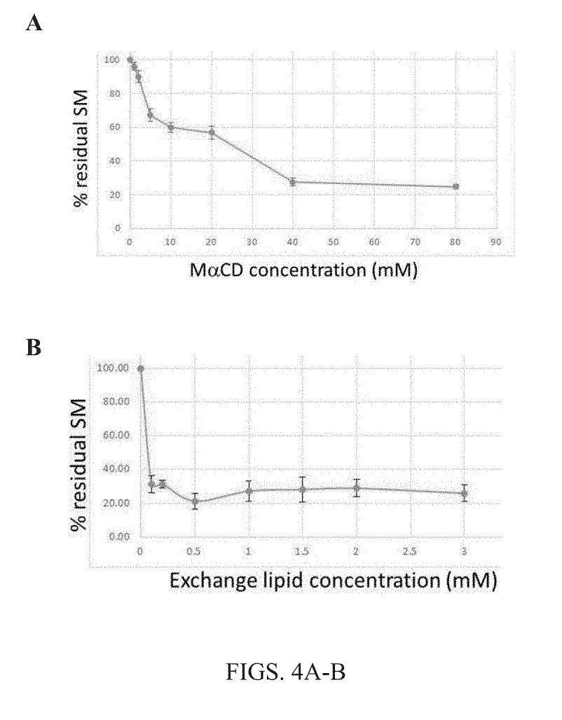

αCD-mediated replacement of plasma membrane outer leaflet lipids with exogenous lipids is efficient as indicated by the radiolabeled SM exchanged out of cells after incubation with a cyclodextrin-lipid complexes was about 70-85% (FIG. 2A and FIGS. 4A-B). This maximum value was not affected when MαCD concentration was increased above 40 mM or exogenous lipid concentrations were varied (FIGS. 4A-B). A slightly higher level of endogenous SM removal by exchange (˜80%) was measured using mass spectrometry (Table 1). As shown in FIGS. 7A and 7B, levels of endogenous SM exchange were the same across all cell types tested (FIG. 7A), and for all lipids exchanged, i.e., bSM, POPC, bSM/POPC or an eSM/DOPC mixture (FIG. 7B).

Trypsinization of cells prior to exchange did not increase exchange levels, indicating exchange was not limited by cell-plate contacts. Notably, after a 30 min exchange with cyclodextrin-lipid complexes comprising bSM and MαCD, adding a fresh mixture of cyclodextrin-lipid complexes comprising bSM and MαCD did not result in additional exchange of membrane lipids, as shown in FIG. 2C. This indicates complete exchange of plasma membrane (PM) outer leaflet SM, with the unexchangable cellular portion of SM being inaccessible due to location in the cytosolic leaflet of the cellular membrane or internal membranes.

Fluorescence micrographs set forth in FIG. 3A, show a plasma membrane localization of NBD-lipid exchanged into cells. The location of exchanged, labeled-lipids using the present methods was identified in the outer leaflet. Specifically, when comparing FIG. 3A (left) and (right) one can clearly see that treatment of the cells with a membrane-impermeable NBD-reducing agent, sodium dithionite (FIG. 3A, right) results in the loss of nearly all NBD-mediated fluorescence. Further confirmation by spectroscopic measurements of the decrease in NBD fluorescence upon dithionite treatment show that about 70-80% of NBD-lipid exchanged into cells was accessible to dithionite (FIG. 3B). Hence, the present lipid exchange methods efficiently exchange exogenous, fluorescent-labeled lipids between a cyclodextrin-lipid complex of the present disclosure and the plasma membrane of a living cell.

In addition, exogenous radiolabeled lipids introduced into cells after exchange by the cyclodextrin-lipid complex mediated methods of the present disclosure was efficient and specific to the plasma membrane. As shown in FIG. 6B, 80% of the exogenous ¹⁴C-SM delivered into cells could be removed by a second exchange, showing its distribution was similar to that of the endogenous lipid. This also indicates that SM asymmetry remained stable at least one hour after the initial exchange. FIG. 6C shows measurements of SM content in cells after replacement of outer leaflet lipids with exogenous SM using the present methods. These data confirm that the exchanged lipids remain in the cell membrane for hours.

### Example 4: Selectivity of Lipid Exchange

A549 cells were labeled with ³H and the lipids were extracted and analyzed. The major cell membrane lipids are PS, PI, SM, PC, PE and neutral lipids including cholesterol. To identify the efficiency at which cellular lipids were being exchanged, cell membrane lipids were radiolabeled and measured for residual radioactivity after carrying out the lipid exchange methods of the present disclosure, whereby cyclodextrin-lipid complex included non-radioactive lipids.

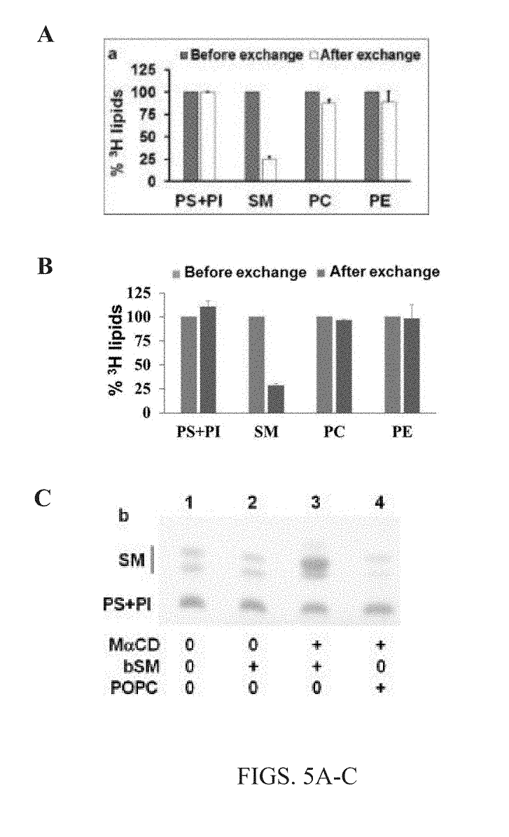

The exchange of lipids other than SM is consistent with exchange being restricted to lipids in the outer leaflet of the cellular membrane. As shown in FIG. 5A, under conditions of maximal SM exchange, % exchange of PC and PE was very low (10-15%), and there was virtually no exchange of PS+PI. These lipids are all either found in high amounts in internal membranes and/or are located in the cytosolic leaflet of the plasma membrane and thus not accessible to exchange. Notably, these findings were not altered by the how radioactivity values were normalized as seen by comparing FIGS. 5A-B. Further, exchange using cyclodextrin-lipid complexes of the present disclosure had no effect on cholesterol or triglyceride levels, which is a major improvement over existing methods, such as MPCD mediated exchange.

Analogous experiments with unlabeled bSM in which lipid levels were estimated by charring of lipid bands on TLC experiments gave similar results as those obtained with radiolabeled lipids. In agreement with radioactivity measurements, most endogenous SM was removed upon exchange with exogenous POPC (FIG. 5C, lane 4), while PS+PI levels were not affected by exchange (FIG. 5C, lanes 3-40). Again, there was no notable exchange of lipids in the absence of cyclodextrin-lipid complexes comprising MαCD (FIG. 5C, lanes 1-2).

Because endogenous SM is ˜40% total outer leaflet lipid, complete 1:1 exchange of outer leaflet lipid with exogenous SM should increase SM concentration in the plasma membrane outer leaflet ˜2.5-fold (if outer leaflet SM is 80% of total SM, total SM should increase by 2.2-fold). This change in SM concentration was observed, as set forth in FIG. 5C, lanes 2-3.

Additionally, mass spectrometry experiments using exchange of the unnatural lipid C_{17:0} SM into cells gave similar results. See Table 1. Specifically, the amount of exogenous C_{17:0} SM in cells after exchange was 2.1-fold higher than the amount of total endogenous cell SM prior to exchange (Table 1), indicative of complete replacement of the plasma membrane outer leaflet phospholipid and sphingolipid in a roughly 1:1 exchange process.

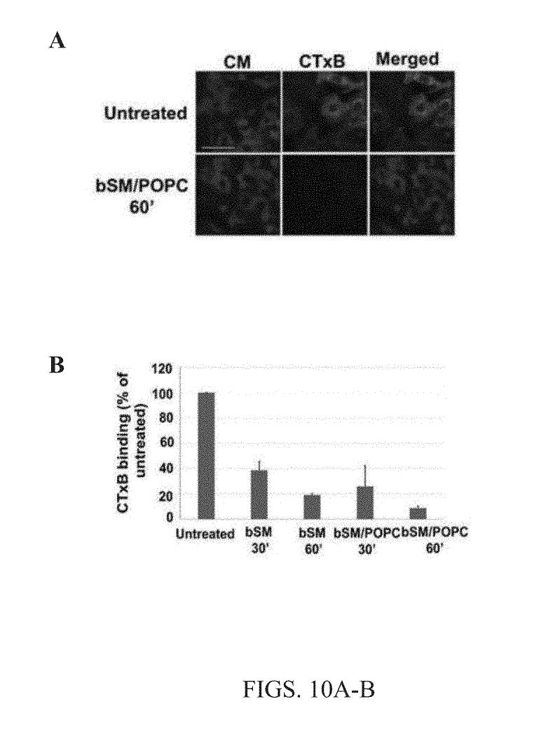

Further, as shown in FIGS. 10A-B, the amount of the endogenous outer leaflet lipid ganglioside (GM1) exchanged out of cells was also consistent with efficient exchange of outer leaflet lipids of the plasma membrane. As assayed by the binding of fluorescently-labeled cholera toxin B subunit (CTxB), ˜90% of CTxB binding was abolished (FIGS. 10A-B). Taken together, the instant methods can be used to exchange outer lipid gangliosides present in cells including, for example, the most common of gangliosides (GM1, GD1a, GM2, and GM2).

Overall, the data provided herein clearly shows that plasma membrane outer leaflet lipids can be substantially remodeled using the present cyclodextrin-lipid complex mediated exchange methods. Notably, each experiment conducted did not negatively affect cell health or growth after membrane lipid exchange despite experimentation over a wide range of conditions.

### Example 5: The Effect of Lipid and MαCD Concentration on SM Exchange Efficiency

FIG. 4A of the present disclosure confirms the dependence of SM exchange efficiency on the αCD concentration. About 70%-80% SM was exchanged between cells and cyclodextrin-lipid complex using 40 mM MαCD and 1.5 mM SM. The cells were healthy and viable after a 1 hour treatment with cyclodextrin-lipid complex at an MαCD concentration of 40 mM or below. Increasing MαCD concentration to 80 mM resulted in slightly more SM exchange. However, at a concentration of 80 mM cell health and viability declined.

In contrast, as shown in FIG. 4B altering lipid concentration had little effect on the efficiency of SM exchange. Here, the formation of cyclodextrin-lipid complexes including bSM and MαCD over the concentration range recited reveals a consistent amount of residual exogenous membrane lipid for all lipid concentrations above 0.2 mM.

### Example 6: Exchange of Lipids in Different Cell Lines

As indicated above and shown in FIG. 7A, the present method for the exchange of endogenous cell membrane lipids is efficient and applicable for numerous different types of cells. For example, the amount of endogenous SM exchanged with exogenous lipids bound to cyclodextrin-lipid complexes of the present disclosure observed across kidney (COS-7), breast (MDA-MB-231), epithelial (A549) and pancreatic (BxPC-3) cells was between 60 and 70%. The slightly lesser exchange in BxPC-3 cells may reflect the fact that pancreatic cell membranes contain a smaller fraction of endogenous SM in their plasma membrane outer leaflets.

### Example 7: SM Exchange with Different Combination(s) of Lipids

As shown in FIG. 7B and Table 4, a similar amount of endogenous lipid is exchanged using the present methods regardless of the type of exogenous cell membrane lipid bound to the cyclodextrin-lipid complex. More specifically, a 1.5 mM concentration of either SM, POPC, DOPC or a combination thereof results in exchange of over 70% of endogenous membrane lipid. More specifically, Tables 3 and 4 showing mass spectrometry experiments using exchange of exogenous C_{17:0} SM into cells, reveal a high level of exchange for all species (Table 4), the ratio for exchange for long SM (with no less than 36 carbons) relative to short acyl chain SM (with less than 36 carbons) increases. These results demonstrate that using the present methods, the exchange of shorter acyl chains SM species in a plasma membrane are particularly exceptional.

### Example 8: Exemplary Methods and Instructions for Inclusion in Kits of the Present Disclosure for the Exchange of Lipids in Living Mammalian Cells

All steps carried out at room temperature unless otherwise noted.

Preparation of MαCD stock solution. MαCD solid from AraChem (The Netherlands, CDexA-066) is dissolved in water or in DPBS (GIBCO, 14190-144). Typically, 6.4 g MαCD dissolved in 12 ml DPBS to make a roughly 380 mM MαCD stock solution. The MαCD stock solution may be slightly turbid, if so, the solution is filtered using a BD 10 ml syringe (BD, 309604) equipped with a 0.2-μm pore filter (SARSTEDT, Ref 83.1826.001). Turbidity is removed by the filtration.

The final stock solution concentration in water can be measured directly by dry weight. Here, aluminum foil is cut into squares, approximately 15 mm to a, side, numbered on the exterior surface, and then shaped into a liquid-tight container. Using forceps, the foil container is weighed to the closest microgram, and the weight is recorded for each container. 10 μl of MαCD solution is added into each container. For best accuracy 3-5 such samples are prepared and quantified. The solution is dried under a gentle stream of nitrogen (<2 psi) for 10 min, and the containers are placed in a high vacuum for at least 45 min to remove residual water until drying is complete. Next, the containers are then reweighed, and the difference in weight is used to calculate the concentration of the solution.

In the alternative, when MαCD sample are dissolved in in DPBS, concentration is measured by comparison to a standard curve. Here, the index of refraction of MαCD/DPBS as measured using, for example, a Bausch and Lomb refractometer is compared to that for a, standard curve of MαCD calibrated by dry weight.

Preparation and measurement of concentration of lipid stock solutions. Lipid, e.g., bSM, is provided as a lyophilized solid or dissolved in chloroform (Avanti, 860062C or 860062P). If solid, it is dissolved in chloroform. All, lipid solutions are stored in glass at −20° C. or −70° C. The lipid solution is gently warmed in a hot block to dissolve any lipid precipitate. Aluminum foil is cut into squares, approximately 15 mm to a side, numbered on the exterior surface, and then shaped into a liquid-tight container. Using forceps, the aluminum containers are weighed (e.g. using a Calhn, C-33 microbalance) to the closest microgram, and the weight of each container is recorded. 10 μl warmed lipid solution is added into the container using, for example, a positive displacement pipet with a glass bore (e.g., Drummond brand digital microdispensers). For best accuracy, 3-5 such samples are prepared. The lipid solution is dried under a gentle stream of nitrogen (<2 psi) for 10 min and containers are placed in a high vacuum for at least 45 min to remove the remaining solvent until drying is complete. The containers are then reweighed, and the difference in weight is used to calculate the concentration of the solution.

Preparation of lipid containing multilameliar vesicles (MLIV). The desired amount of lipid is extracted from a lipid stock solution, as set forth herein. Specifically, an amount of the stock solution is placed in a container lined with aluminum foil and the lipid is dried under a gentle stream of nitrogen (<2 psi) until no liquid is seen and drying is complete. At the same time, RPMI 1640 medium (without serum) in a 50 ml Falcon brand conical centrifuge tube is warmed in a water bath. To make MLV, the desired volume of RPMI 1640 medium without serum is added into the glass tube containing the dried lipid to give 2-times the final desired lipid concentration (e.g., 3 mM when the cells are to be incubated with 1.5 mM lipid) and vortexed briefly. The glass tube is sealed with Teflon tape (SP Scienceware) and incubated in a 70° C. water bath for 5 min, vortexing briefly every minute.

Preparation of Lipid-Loaded αCD for Lipid Exchange Experiments in Cells. The desired amount of MαCD (from a stock solution of MαCD dissolved in DPBS) is mixed with RPMI 1640 medium with serum (to give a concentration of 80 mM MαCD). Then the MαCD solution is mixed with an equal volume of MLV solution prepared as described above, and added into a 15 ml conical centrifuge tube. The mixture is vortexed briefly. To generate the cyclodextrin-lipid complexes of the present disclosure, the mixture is placed in a 37° C. incubator for 30 min.

Preparation of A549 Human Lung Cancer Cells for Exchange. One day before lipid exchange, the A549 cells are split. For example, A549 cells in a 100% confluent 10 cm plate are equally split into three 10 cm plates (Falcon, 353003) and are cultured in 10 mL RPMI 1640 supplemented with 10% FBS. The cells are then incubated in 5% CO₂ incubator at 37° C. for 16-24 hrs. Cell confluence is checked visually by microscopy. Plates with 90-100% confluent cells are then used for lipid exchange.

Exchange of Lipids between A549 Cells and Cyclodextrin-lipid Complexes. Growth medium is removed from cells after splitting and the cells are washed three times with 10 ml DPBS, and the wash liquid is removed by aspiration. A 1500 μL aliquot of cyclodextrin-lipid complexes including MαCD with 1.5 mM bSM is added onto the above A549 cells. The cells are incubated with the MαCD-lipid complexes for 1 h at 37° C. in a 5% CO₂ incubator with rocking every 10 minutes. After the 1 hour incubation the cyclodextrin-lipid complex solution is removed and placed into a 1.5 ml plastic centrifuge tube (Beckman Coulter, 357448 for future analysis of the lipids exchanged. The cells are washed three times with 10 ml DPBS and then removed by scraping in 5 mL DPBS. The cells are then centrifuged for 3 minutes at about 2000×g (e.g., 1725×g in Savant Speedvac concentrator (Savant, Hicksville, N.Y.) to form a pellet. The DPBS liquid solution is gently aspirated, and the cells (pellet) are saved for lipid analysis.

Lipid Extraction and Analysis of A549 Cell Lipids. Lipids can be extracted using many known methods. Specific extraction methods include the following: A) Hexane-isopropanol method that includes, mixing 1.8 ml mixed hexanes and 1.2 ml isopropanol in the glass tube with the A549 cells. The tube is vortexed to mix the cells and solvent. The tube is incubated for 30 minutes, vortexing every 5 minutes. The sample is centrifuged for 5 minutes at about 2000×g to precipitate cell debris. The organic solvent layer contains the lipids and is removed and placed in a new glass tube. The removed solvent is dried with nitrogen gas while gently warming at 50° C., or B) Chloroform-methanol method that includes mixing 2 ml chloroform and 1 ml methanol into the glass tube with the A549 cells. The tube is vortexed to mix the cells and solvent. The tube is incubated for 30 minutes, vortexing every 5 minutes. After 30 minutes, 600 μl of 0.9% NaCl is added to the solution and vortexed. The sample is centrifuged for 5 minutes at about 2000×g. The bottom phase containing the lipid in organic solvent is removed and placed in a new glass tube. The organic solvent is dried with nitrogen gas while gently warming at 50° C.

Separation and Analysis of lipids exchanged out from A549 cells. The MαCD cyclodextrin-lipid complex containing supernatant that is isolated after lipid exchange (above) is collected and subject to ultracentrifugation at 72,000 rpm in an Optima™ TL Ultracentrifuge using a TLA 100.3 rotor (Beckman Coulter, Fullerton, Calif.) for 30 minutes to remove cells/cell debris. The remaining mixture is removed to a glass tube, and an equal volume of 3:2 (v:v) hexane/isopropanol is added to the mixture. The mixture is vortexed briefly. The tube is then incubated for 30 minutes with vortexing every 5 minutes. The mixture was partitioned into aqueous and solvent phase by centrifugation at 2000×g for about 30 min. The top lipid-containing solvent phase was removed and placed in a new glass tube. This step was repeated if necessary. The lipid-containing solvent phase is dried completely with nitrogen and ready for analysis.

Instructions for identifying optimal lipid exchange conditions for mammalian cells. To screen for optimal MαCD concentrations before carrying out lipid exchange 1.5 mM lipid mixed with a series of MαCD solutions having different MαCD concentrations (e.g., 0, 5, 10, 15, 20, 25, 30, 35, 40, 45, 50, 55, 60 mM) are incubated according to the present methods to form MαCD cyclodextrin-lipid complexes of the present disclosure. The MαCD cyclodextrin-lipid complexes at all concentrations are added to cells to determine the highest MαCD concentration possible with no cell rounding for the maximum amount of time. Here, cells are split into a 12-well plate one day before use thereof. For example, cells from a 100% confluent well are trypsinized and then resuspended into 30 ml culture medium. A 1 ml aliquot is added into each well. The next day cells will be 90-100% confluent. Next, 300 μL MαCD cyclodextrin-lipid complex solutions are prepared with 1.5 mM bSM and various concentrations of MαCD (e.g., 0, 5, 10, 15, 20, 25, 30, 35, 40, 45, 50, 55, 60 mM MαCD) as described above. Each cyclodextrin-lipid complex mixture is incubated at 37° C. for 30 min. Cells are washed 3-times in PBS, as described herein and 1500 uL of each MαCD) cyclodextrin-lipid complex solutions is added into a separate well and incubated for 1 h at 37° C. in a 5% CO₂. The cell condition is checked by microscope every 15 minutes. The specific concentration mixture is acceptable for lipid exchange if no cell rounding is observed during the incubation period.

TABLE 1
Effect of lipid exchange upon cell phospholipid
and sphingomyelin content.

| | % of total (endogenous) lipids^{a} | | % of total endogenous lipids^{a} | |
|---|---|---|---|---|
| | in cell before exchange | | in cells after exchange^{b} | |
| Lipids | MS | Radioactivity | MS^{d} | Radioactivity |
| PS | 11.5 ± 0.8 | 14.3 ± 1.3 ^{c} | 9.9 ± 0.2 | 17.7 ± 2.3 ^{c} |
| PI | 10.5 ± 0.6 | | 13.7 ± 0.9 | |
| SM | 17.7 ± 0.5 | 13.0 ± 0.7 | 3.3 ± 0.0 | 4.1 ± 0.6 |
| PC | 50.7 ± 2.0 | 57.4 ± 2.4 | 60.1 ± 0.2 | 61.6 ± 3.3 |
| PE | 9.6 ± 0.1 | 15.3 ± 1.6 | 11.9 ± 0.4 | 16.7 ± 2.2 |

TABLE 2
Time of incubation of A549 cells with MαCD
at which normal morphology begins to change

| Lipids | [MαCD] | incubation time |
|---|---|---|
| None | 40 mM | ≈15′ |
| 1.5 mM bSM | 40 mM | >6 h |
| 3 mM bSM | 40 mM | ≈4 h |
| 3 mM bSM | 80 mM | >6 h |
| 1.5 mM mSM | 40 mM | >6 h |
| 3 mM mSM | 40 mM | ≈3 h |
| 3 mM mSM | 80 mM | >6 h |
| 1.5 mM eSM | 40 mM | >6 h |
| 3 mM eSM | 40 mM | ≈30′ |
| 6 mM eSM | 40 mM | ≈15′ |
| 3 mM eSM | 80 mM | ≈1 h |
| 6 mM eSM | 80 mM | >6 h |
| 0.75 mM eSM/0.75 mM POPC | 40 mM | ≈4 h |
| 1.5 mM eSM/1.5 mM POPC | 40 mM | ≈2 h |
| 3 mM eSM/3 mM POPC | 40 mM | ≈45′ |
| 5 mM eSM/5 mM POPC | 40 mM | ≈45′ |
| 1.5 mM eSM/1.5 mM POPC | 80 mM | ≈15′ |
| 3 mM eSM/3 mM POPC | 80 mM | ≈3 h |
| 1.5 mM POPC | 40 mM | ≈15′ |
| 3 mM POPC | 40 mM | ≈45′ |
| 6 mM POPC | 40 mM | ≈45′ |
| 3 mM POPC | 80 mM | ≈15′ |

TABLE 3
Percentage of abundant (>1%) phospholipids in cells
before and after exchange with C₁₇:₀ SM-MαCD.

| | % of lipid | % of lipid | | | |
|---|---|---|---|---|---|
| | species in | species in | Ratio | Group | |
| | cells before | cells after | before/ | Aver- | P |
| | exchange | exchange | after^{a} | age | value |
| PC | | | | | |
| species | % of total PC | | | | |
| C32:1 | 14.23 ± 0.58 | 14.80 ± 0.70 | 0.96 | 0.99 ± 0.11 | 0.001 |
| C34:1e | 4.07 ± 0.02 | 3.53 ± 0.28 | 1.15 | | |
| C34:1 | 31.35 ± 0.75 | 30.21 ± 0.30 | 1.04 | | |
| C36:1 | 1.87 ± 0.02 | 1.68 ± 0.30 | 1.11 | | |
| C32:2 | 2.24 ± 0.23 | 2.47 ± 0.30 | 0.91 | | |
| C34:2 | 9.16 ± 0.11 | 11.00 ± 0.19 | 0.83 | | |
| C36:2 | 11.24 ± 0.16 | 11.82 ± 0.59 | 0.95 | | |
| C38:2 | 1.44 ± 0.06 | 0.53 ± 0.05 | 2.7 | | |
| C34:3 | 0.87 ± 0.02 | 1.29 ± 0.13 | 0.67 | 0.74 ± 0.10 | |
| C36:3 | 2.08 ± 0.22 | 3.22 ± 0.23 | 0.65 | | |
| C36:4 | 2.59 ± 0.04 | 3.66 ± 0.17 | 0.71 | | |
| C36:4e | 2.34 ± 0.10 | 2.92 ± 0.17 | 0.8 | | |
| C38:4 | 1.37 ± 0.03 | 1.47 ± 0.14 | 0.93 | | |
| C38:5 | 1.85 ± 0.01 | 2.35 ± 0.01 | 0.79 | | |
| C38:6 | 0.83 ± 0.07 | 1.25 ± 0.06 | 0.66 | | |
| PE | | | | | |
| Species | % of total PE | | | | |
| C34:1 | 10.34 ± 0.75 | 9.80 ± 0.50 | 1.06 | 1.08 ± 0.05 | 0.68 |
| C34:2 | 4.27 ± 0.01 | 3.73 ± 0.02 | 1.14 | | |
| C36:2 | 17.27 ± 2.17 | 16.49 ± 2.25 | 1.05 | | |
| C36:3 | 2.08 ± 0.12 | 1.93 ± 0.20 | 1.08 | 1.12 ± 0.24 | |
| C36:3p/ | 1.46 ± 0.14 | 1.01 ± 0.12 | 1.44 | | |
| C38:3 | 2.68 ± 0.29 | 2.92 ± 0.32 | 0.92 | | |
| C36:4 | 5.67 ± 0.31 | 5.66 ± 0.05 | 1 | | |
| C36:4p/ | 1.81 ± 0.04 | 1.21 ± 0.03 | 1.5 | | |
| C38:4 | 32.94 ± 0.03 | 35.76 ± 1.71 | 0.92 | | |
| C38:5 | 11.48 ± 0.28 | 11.32 ± 0.99 | 1.01 | | |
| C40:5 | 1.22 ± 0.18 | 1.30 ± 0.09 | 0.94 | | |
| C38:6 | 1.45 ± 0.05 | 1.52 ± 0.13 | 0.95 | | |
| C40:6 | 2.27 ± 0.36 | 2.28 ± 0.28 | 0.99 | | |
| C40:7 | 1.43 ± 0.13 | 1.46 ± 0.03 | 0.98 | | |
| PI | | | | | |
| species | % of total PI | | | | |
| C36:2 | 6.80 ± 0.63 | 8.48 ± 1.33 | 0.8 | 0.8 | NA |
| C36:4 | 5.72 ± 0.71 | 4.67 ± 0.44 | 1.23 | 1.07 ± 0.15 | |
| C38:4 | 66.05 ± 6.88 | 70.05 ± 0.11 | 0.94 | | |
| C38:5 | 14.54 ± 2.79 | 14.10 ± 0.93 | 1.03 | | |
| PS | | | | | |
| species | % of total PS | | | | |
| C34:1 | 23.90 ± 0.41 | 24.94 ± 0.51 | 0.96 | 1.00 ± 0.08 | 0.15 |
| C36:1 | 40.45 ± 0.14 | 36.44 ± 0.24 | 1.11 | | |
| C34:2 | 2.73 ± 0.16 | 2.80 ± 0.07 | 0.97 | | |
| C36:2 | 13.78 ± 0.08 | 15.06 ± 0.76 | 0.91 | | |
| C38:2 | 1.93 ± 0.06 | 1.83 ± 0.10 | 1.05 | | |
| C36:3 | 1.38 ± 0.22 | 1.73 ± 0.14 | 0.8 | 0.90 ± 0.11 | |
| C38:3 | 2.62 ± 0.24 | 2.45 ± 0.07 | 1.07 | | |
| C38:4 | 9.09 ± 0.08 | 9.67 ± 0.36 | 0.94 | | |
| C40:4 | 1.10 ± 0.32 | 1.22 ± 0.26 | 0.9 | | |
| C40:6 | 1.26 ± 0.09 | 1.59 ± 0.05 | 0.79 | | |

TABLE 4
Percentage of sphingomyelin species in untreated cells and in cells after
C₁₇ SM-MαCD exchange. P values < 0.001

| | % of total | | | | % of total | | | |
|---|---|---|---|---|---|---|---|---|
| | SM in | % of total | | | lipids in | % of total | | |
| | cells | SM in | | | cells | lipids in | | |
| | before | cells after | | Group | before | cells after | | Group |
| SM | exchange | exchange | Ratio 1 | avg. | exchange | exchange | Ratio 2 | avg. |
| C32:1 | 2.48 ± 0.10 | 1.44 ± 0.17 | 1.73 | 1.83 ± 0.19 | 0.44 ± 0.00 | 0.03 ± 0.00 | 14.86 | 15.78 ± 1.65 |
| C34:1 | 61.84 ± 2.43 | 35.93 ± 2.39 | 1.72 | | 10.96 ± 0.10 | 0.74 ± 0.04 | 14.8 | |
| C34:2 | 3.86 ± 0.02 | 1.88 ± 0.32 | 2.05 | | 0.68 ± 0.02 | 0.04 ± 0.01 | 17.68 | |
| C36:1 | 3.36 ± 0.14 | 6.53 ± 0.78 | 0.52 | 0.4 ± 0.11 | 0.60 ± 0.04 | 0.13 ± 0.01 | 4.44 | 3.45 ± 0.93 |
| C42:1 | 3.13 ± 0.89 | 10.35 ± 2.07 | 0.3 | | 0.56 ± 0.18 | 0.21 ± 0.05 | 2.6 | |
| C42:2 | 16.83 ± 3.56 | 43.87 ± 1.59 | 0.38 | | 2.99 ± 0.72 | 0.90 ± 0.05 | 3.31 | |

What is claimed is:

1. A method for substituting lipids in a cell membrane comprising:
providing a, cell, wherein said cell comprises a cellular membrane with a lipid bilayer;
forming a cyclodextrin-lipid complex comprising at least one lipid bound to a cyclodextrin; and
incubating said cell and said cyclodextrin-lipid complex in solution such that at least one lipid is exchanged between said lipid bilayer of said cell and said cyclodextrin-lipid complex.

2. The method of claim 1, wherein said cyclodextrin is an α-cyclodextrin.

3. The method of claim 2, wherein said cyclodextrin is a methyl-α-cyclodextrin.

4. The method of claim 1, wherein said incubation results in the exchange of at least 60% of lipids in an outer leaflet of said lipid bilayer of said cell.

5. The method of claim 4, wherein said incubation results in the exchange of at least 70% of lipids in an outer leaflet of said lipid bilayer of said cell.

6. The method of claim 1, wherein said incubation occurs for a duration of between 30 minutes and 2 hours.

7. The method of claim 6, wherein said duration is for 1 hour.

8. The method of claim 1, wherein said cyclodextrin-lipid complex comprises a lipid selected from the group consisting of a phospholipid and a sphingolipid.

9. The method of claim 8, wherein said lipid is sphingomyelin or phosphatidylcholine.

10. The method of claim 8, wherein said lipid is an unnatural lipid or comprises a label.

11. The method of claim 10, wherein said label is selected from the group consisting of a fluorescent dye and a radio-isotope.

12. The method of claim 1, further comprising forming a multilamellar vesicle comprising at least one lipid prior to forming said cyclodextrin-lipid complex.

13. The method of claim 12, wherein forming said cyclodextrin-lipid complex comprises incubating said multilamellar vesicle with a solution comprising a cyclodextrin.

14. The method of claim 13, wherein said incubation occurs at about 37° C. for about 30 minutes.

15. The method of claim 1, wherein said cell is a living cell.

16. The method of claim 1, wherein said cyclodextrin is not a beta-cyclodextrin (β cyclodextrin).

17. A composition for substituting membrane lipids comprising:
at least one α-cyclodextrin; and
at least one lipid, wherein said at least one lipid is bound to said at least one α-cyclodextrin.

18. The composition of claim 17, wherein said α-cyclodextrin is a methyl-α-cyclodextrin.

19. The composition of claim 18, wherein said at least one lipid is selected from the group consisting of a phospholipid, a sphingolipid and combinations thereof.

20. The composition of claim 17, wherein said at least one lipid comprises sphingomyelin, phosphatidylcholine or a combination thereof.

21. The composition of claim 17, wherein said at least one lipid is an unnatural lipid.

22. The composition of claim 21, wherein said unnatural lipid is N-hepadecanoyl-D-erythro-sphingosylphosphorlcholine (C_{17:0} SM).

23. The composition of claim 17, wherein said at least one lipid comprises a label.

24. The composition of claim 23, wherein said label is selected from the group consisting of a fluorescent dye and a radioisotope.

25. The composition of claim 17, wherein said at least one lipid is bound to a hydrophobic interior portion of said at least one α-cyclodextrin.

26. A kit for substituting lipids in a cellular membrane comprising;
at least one α-cyclodextrin;
at least one lipid
instructions for forming a cyclodextrin-lipid complex comprising said least one lipid bound to said α-cyclodextrin; and
instructions describing a method for using said at least one cyclodextrin-lipid complex to exchange said at least one lipid between a lipid bilayer of said cell membrane and said cyclodextrin-lipid complex.

27. The kit of claim 26, further comprising a sample of cells.

28. The kit of claim 26, wherein said at least one α-cyclodextrin is a methyl α-cyclodextrin.

29. The kit of claim 26, further comprising a sample of a fluorescent dye, and instructions for labeling said at least one lipid of with said fluorescent dye.

30. The kit of claim 26, wherein said kit further comprises instructions for labeling said at least one lipid with a radio-isotope, and methods for detecting the presence of said labeled lipid.

31. The kit of claim 26, wherein said at least one lipid is selected from the group consisting of a phospholipid, a sphingolipid and combinations thereof.

32. The kit of claim 26, wherein said at least one lipid comprises sphingomyelin, phosphatidylcholine or a combination thereof.

33. The kit of claim 26, wherein said at least one lipid comprises an unnatural lipid.

34. The kit of claim 33, wherein said unnatural lipid is N-hepadecanoyl-D-erythro-sphingosylphosphorylcholine (C_{17:0} SM).

35. The kit of claim 26, further comprising instruction for forming a multilamellar vesicle comprising said at least one lipid.

36. The kit of claim 35, wherein said instructions for forming said cyclodextrin-lipid complex comprises incubating said multilamellar vesicle with a solution comprising said at least one α-cyclodextrin.
